(19) United States
(12) Patent Application Publication
(10) Pub. No.: US 2009/0054713 A1
(43) Pub. Date: Feb. 26, 2009

(54) Method for preparing polyolefinic bases of synthetic oils

(76) Inventors: Petr Evgenievich Matkovsky, Chernogolovka (RU); Sergei Mikhailovich Aldoshin, Chernogolovka (RU); Vladimir Nikolaevich Troitsky, Chernogolovka (RU); Galina Pavlovna Startseva, Chernogolovka (RU); Valery Ivanovich Savchenko, Chernogolovka (RU); Mikhail Alexandrovich Demidov, Nizhnekamsk (RU); Vladimir Garafovich Hhamsutdinov, Nizhnekamsk (RU); Gabbas Lukmanovich Ilyasov, Nizhnekamsk (RU)

Correspondence Address: LADAS & PARRY LLP, 26 WEST 61ST STREET, NEW YORK, NY, 10023, US

(21) Appl. No.: 11/792,908

(22) PCT Filed: Dec. 21, 2005
(86) PCT No.: PCT/RU05/00655

§ 371 Date: Dec. 28, 2007

(30) Foreign Application Priority Data
Dec. 22, 2004 (RU) 2004137372

(51) Int. Cl. C07C 2/08

(52) U.S. Cl. 585/326

(57) ABSTRACT

The invention relates to a method for the preparation of the polyolefinic bases of synthetic oils by the cationic oligomerization of olefinic feedstock and can be used in the petrochemical industry. A new method for the preparation of the polyolefinic bases of synthetic oils has been developed, which comprises the steps of conditioning olefinic feedstock, preparing and dosing in a reactor, solutions and suspension of the components of a catalytic system Al(O)—HCl-TBCh, isomerizing alpha-olefins and oligomerizing higher olefins and mixtures thereof under the action of the catalytic system Al(O)—HCl-TBCh, separating spent catalyst, dividing an oligomerizate into fractions and hydrogenating the fractions separated under the action of a catalyst Pd(0.2% w)/A1203+NaOH. The invention provides the improvement of all steps of the method elaborated. For the corrosive activity of products to be removed, a method further comprises a step of the dechlorination of oligomerizate present chlorine-containing oligoolefins with metallic aluminum, triethyl aluminum, alcohol KOH solutions or the thermal dehydrochlorination of chlorine-containing polyolefins in the absence or presence of KOH. For improvement of method technico-economic indices owing to an increase in the yield of polyolefin target fractions having a kinetic viscosity of 2-8 cSt at 100° C., the method further comprises a step of the thermal depolymerization of restricted consumable high molecular polyolefins with a kinetic viscosity of 10-20 cSt at 100° C. to target polyolefins with a kinetic viscosity of 2-8 cSt at 100° C.

### FIELD OF THE INVENTION

The invention relates to the petrochemical technologies, namely a method for the preparation of the polyolefinic bases of synthetic oils through the cationic oligomerization of olefinic feedstock and can be used in the petrochemical industry.

The products obtained according to the method sought to be protected can be used as a base of synthetic polyolefinic (oligo-olefinic) oils of diverse designated purposes: motor (automobile, aviation, helicopter, tractor, tank); transmission, reductor, vacuum, compressor, refrigerator, transformer, cable, spindle, medical, in the composition of various lubricants as well as plasticizers for plastics, rubbers, solid propellants; starting materials for the preparation of dopants, emulsifiers, flotation agents, foaming agents, components of cooling lubricant and hydraulic fluids; high-octane additives to fuels, to mention only few.

### PRIOR ART

Known in the art are methods for preparing the polyolefinic bases of synthetic oils through the cationic oligomerization of higher olefins, which comprise a step of conditioning olefinic feedstock and solutions of the components of a catalytic system, a step of oligomerization of olefinic feedstock, a step of releasing from an oligomerizate, spent catalyst by a method of water-alkali and subsequent water washing-off, a step of separation of a purified oligomerizate into fractions and a step of hydrogenation of the target fractions separated out.

Well-known methods for preparing the polyolefinic bases of synthetic oils appreciably differ from one another in the compositions of cationic catalysts to be used therein.

In accordance with conventional methods, the cationic oligomerization of olefins C₃-C₁₄ (i.e. olefins containing from 3 to 14 carbon atoms) is initiated (catalyzed), using: proton acids (Brendsted acids); aprotic acids (Lewis acids); alkylaluminum—(or boron) halides; salts of stable carbocations R⁺A⁻; natural and synthetic alumosilicates, zeolites or heteropoly acids in H-form; different binary and ternary complexes comprising monomers; polyfunctional Ziegler-Natta catalysts; metallocene catalysts; the physical methods of stimulation of chemical reactions /1. J. Kennedy “Olefin Cationic Polymerization”, Moscow: MIR Publishers, 1978, 430 pages; 2. J. P. Kennedy, E. Marechal “Carbocationic Polymerization”, N.-Y., 1982, 510 pages/. The widest variety of industrial application as catalysts of the cationic oligomerization of olefins and other monomers is characteristic of catalytic systems including Lewis acids (BF₃, AlCl₃, AlBr₃, TiCl₄, ZrCl₄, etc.), alkylaluminum—(or boron) halides RₙMX₃₋ₙ (wherein R-alkyl C₁-C₁₀—, aryl-, alkenyl- and other groups; M-Al or B; X—Cl, Br, I) and natural or synthetic alumosilicates, zeolites and heteropoly acids in H-form. At the time of preparing motor PAOM based on linear alpha-olefins (LAO) C₆-C₁₄ (predominantly based on decene-I) use is normally made of catalytic systems including Lewis acids or alkylaluminum halides.

Known is a great number of methods for the preparation of the polyolefinic bases of synthetic oils, in accordance with which the LAO C₆-C₁₄ oligomerization catalysts used are represented by systems comprising BF₃ and different proton-donating cocatalysts—water, alcohols, carboxylic acids, carboxylic acid anhydrides, ketones, polyols, and mixtures thereof /1/ Patent U.S. Pat. No. 5,550,307, Aug. 27, 1996. Int. cl. CO7 C 2/14; Nat. cl. 585/525/. The polyolefinic bases of synthetic oils, according to these methods, are obtained by the oligomerization of olefins C₆-C₁₄ under the action of said boron-fluoride catalysts at temperatures between 20 and 90° C. in bulk for 2-5 hours. The concentration of BF₃ in reaction media is varied in the range of from 0.1 to 10% w. The conversion of initial olefins ranges from 80 to 99% w. As a result of oligomerization, for example decene-I, there forms a mixture of di-, tri-, tetramers and more high molecular oligomers. The total content of di- and trimers in products is changed in the range of from 30 to 70% w.

A major disadvantage of all methods for the preparation of synthetic oil polyolefinic bases of this type is the fact that they are based on the use of catalysts comprising deficient, highly volatile, toxic, corrosive active BF₃. Besides, because of a relatively low activity of catalysts of this type in LAO oligomerization, a process proceeds for 2-5 hours. With industrial realization of these methods, use is made of expensive, large in volume and specific quantity of metal mixing reactors in anti-corrosion modification.

Also known /2/ Patent U.S. Pat. No. 5,196,635 of Mar. 23, 1993. Int. cl. C07 C 2/22; Nat. cl. 585-532/ is a great number of methods for preparing the polyolefinic bases of synthetic oils, in accordance with which olefin oligomerization is carried out under the action of cationic catalysts comprising aluminum halides and proton-donating means—water, alcohols, carboxylic acids, ethers or esters, ketones (for example, dimethyl ethylene glycol ether, ethylene glycol-diacetate), alkyl halides /2/ /Patent U.S. Pat. No. 5,196,635 of Mar. 23, 1993. Int. cl. C07 C 2/22; Nat. cl. 585-532/. In some methods, these catalysts are used in combination with nickel compounds /3/ /Patent U.S. Pat. No. 5,489,721 of Feb. 6, 1996. Int. cl. C07 C 2/20; Nat. cl. 585-532/. Nickel compound additives usable in the catalysts in accordance with these methods render possible regulation of the fractional makeup of oligo-olefins to be obtained.

The preparation of the polyolefinic bases of synthetic oils by the oligopolymerization of alpha-(C₄-C₁₄) or inner C₁₀-C₁₅ olefins (obtained by paraffin dehydrogenation) according to method /4/ /Patent U.S. Pat. No. 4,113,790 of Sep. 12, 1978. Int. cl. C07 C 3/10; Nat. cl. 585-532/ is carried out under the action of catalysts AlX₃+protonodonor at temperatures between 100 and 140° C. for 3-5 hours. AlX₃ concentration is varied in the range of from 0.1 to 10 mole %, as calculated for olefins, a protonodonor /Al mole ratio is varied in the range of from 0.05 to 1.25. With an increase in this ratio from 0.05 to 1.25, olefin conversion is reduced from 99 to 12% w.

Methods of this type are characterized by the following general defects:
complicated procedure for the preparation of catalysts comprising a lot of operations—sublimation and grinding of AlCl₃, the preparation of a complex;
catalysts obtained by these methods are viscous, sticky materials, poorly soluble in olefins; because of high adhesion to the cooled walls of reactors they are difficulty discharged from the reactors upon completion of oligomerization;
low activity of the catalysts usable during oligomerization, a factor that dictates the need for the use of large in volume specific quantity of metal mixing reactors;
high consumption coefficients in terms of AlX₃, as calculated for obtainable products.

A major general disadvantage of methods of this type is the fact that their use leads to preparing primarily high-molecular and highly viscous products including up to 1% w chlorine.

There have been developed several methods for the preparation of synthetic oil polyolefinic bases based on the use of bifunctional complex catalysts comprising transition metal compounds (TiCl₄, ZrCl₄) and alkylaluminum halides RₙAlX₃₋ₙ (cf. /5//Patent GB 1522129. Int. cl. C07 C 2/22. Nat. cl. C3P; B /usable according to these methods, in bifunctional catalyst systems of the TiCl₄—RₙAlX₃₋ₙ type, with the formation of two types of active centers—cationic and anionic coordination, owing to which fact the oligomerization of olefins C₃-C₁₄ under the action of the active cationic centers is substantially attended in all cases by the polymerization of olefins C₃-C₁₄ under the action of the anionic-coordination active centers to insoluble high-molecular polyolefins which are removed from the reactor with difficulty. And high-molecular highly-viscous oligo-olefins are formed in all cases under the action of bifunctional complex catalysts, which cannot be used as a base of engine oils finding the widest variety of application thereof. This is a major defect of methods of this type.

In accordance with some methods, the cationic processes of polymerization, oligomerization and alkylation widely used are also two component soluble monofunctional catalytic systems including alkylaluminum halide RₙAlX₃₋ₙ and organohalide R′X, with the mole ratio of R′X/RₙAlX₃₋ₙ=1.0-5.0 (wherein R—CH₃, C₂H₅, C₃H₇ or iso —C₄H₉; X— chloro, bromo or iodo; n=1.0; 1.5 or 2.0; R′—H /6/ /Patent U.S. Pat. No. 4,952,739, Aug. 28, 1990. Int. cl. C07 C /18; Nat. cl. 585/ 18; 585/511/, a primary, secondary or tertiary alkyl, allyl or benzyl /7/ /Patent FRG 2304314, 1980. Int. cl. C08 F 110/20/. In the catalytic systems of this type, RₙAlX₃₋ₙ is a catalyst base and R′X— cocatalyst.

In accordance with these methods, catalytic systems RₙAlX₃₋ₙ—R′X are used for initiating the cationic oligomerization or individual or mixtures of linear alpha-olefins from propylene to tetradecene inclusively to the polyalpha-olefinic bases of synthetic oils in the atmosphere of initial olefins or their mixtures with oligomerization products and paraffin, aromatic or halogen-containing hydrocarbons at temperatures of up to 250° C.

Cationic active centers [R′⁺(RₙAlX₄₋ₙ)⁻] and R′⁺) in the catalytic systems RₙAlX₃₋ₙ—R′X form in line with the following simplified diagram: RₙAlX₃₋ₙ+R′X

[symbol]

[R′⁺(RₙAlX₄₋ₙ)^{-]} [symbol] ^{R′}⁺+(RₙAlX₄₋ₙ)⁻

The formation of cationic active centers in the catalytic systems under consideration occurs at very high velocity, owing to which fact right after mixing the component solutions of said catalytic systems a high concentration of the active cationic centers is achieved and an oligomerization process proceeds without an induction period at very high starting velocity. And the 95/98% conversion of initial olefins to oligomer products at temperatures between 20 and 200° is achieved in six-one minutes, respectively. Such a nature of the kinetics of oligomerization of linear alpha-olefins (LAO) under the action of catalytic systems under consideration provides the possibility to conduct the oligomerization process under high-speed isothermal conditions in tubular type displacement reactors, with a residence time of between I and 10 minutes /8/ /Patent RF 2201799. Sep. 29, 2000. Int. cl. 7 B 01 J 8/06, C 08 F 10/10; Bulletin of Inventions. 2003. No. 10/.

On preparation of the oligo-olefinic bases of synthetic oils in accordance with these methods during LAO oligomerization in bulk or in the atmosphere of paraffin hydrocarbons under the action of catalytic systems RₙAlX₃₋ₙ—R′X there form highly branched oligomers solidifying at low temperatures, comprising one di-, tri- or tetraalkyl substituted double bond and up to 0.2% w monochloroligo-olefins and with oligomerization in the atmosphere of or in the presence of aromatic hydrocarbons (benzene, toluene, naphthalene) there form oligoalkyl aromatic greasy products (telomers) having no double bonds /9/ Patent RF 2199516 of Apr. 18, 2001. MKH 7 CO7C 2/22. Bulletin of Inventions No 6, Feb. 27, 2003.

A major disadvantage of methods for preparing the oligo-olefinic bases of synthetic oils through olefin oligomerization under the action of catalytic systems RₙAlX₃₋ₙ—R′X is the fact that their use during LAO oligomerization (in particular, decene-I) results in forming predominantly high-molecular products with a wide molecular weight distribution and with the low (below 20% w) content of low-molecular target fractions (dimers and trimers of decene-I).

Another disadvantage of methods based on the use of catalytic systems of this type is the fact that the obtainable dimers of decene-I according to these methods are linear and have, after hydrogenation, a solidification temperature of above −20° C.

For this defect to be removed, i.e. improvement of selectivity and technico-economic factors of a method, there has been elaborated a method for preparing the oligo-olefinic bases of synthetic oils, in accordance to which the non-hydrogenated dimers of decene-I are recycled for cooligomerization thereof with decene-I in widely used tri- and tetramers of decene /10/ Patent U.S. Pat. No. 4,263,467 Apr. 2, 1981/. The cooligomerization of dimers of decene-I (43.8% w in charge) with decene-I (40% w decene-I and 15.4% w decane in charge) according to this method is carried out under the action of system BF₃/SiO₂ (D=0.8-2.0 mm)+H₂O (65 ppm in charge) at temperatures of 15 to 30° C., a pressure of 1 to 6.9 at, with a charge consumption of 2.5 l/hr per liter of the catalyst. The content of dimers of decene-I, on leaving a reactor is diminished from 43.8 to 20.7% w while the content of trimers of decene-I is increased to 41.8% w. This solution makes it possible to skillfully use the non-consumable (solidifying at high temperatures) linear dimers of decene-I. A disadvantage of this solution is a sharp drop in process efficiency based on this method.

A third general disadvantage of all the methods based on the use of catalytic systems RₙAlX₃₋ₙ—R′X is the fact that the usable catalytic systems comprise a fuel spontaneously inflammable in air, hazardous in production, in transportation and in the use of RₙAlX₃₋ₙ organoaluminium compound.

And last but not least, a fourth general disadvantage of all the methods based on the use of catalytic systems RₙAlX₃₋ₙ—R′X is the fact that under the action of these catalytic systems there form products containing up to 1.0% w chlorine in the form of monochloroligo-olefins.

The closest to a method for preparing the oligo-olefinic bases of synthetic oils, in accordance with the present invention, are the methods of cationic polymerization, olefin oligomerization and alkylation of aromatic hydrocarbons with olefins under the action of catalytic systems including metallic aluminum. The latter, as such, is not a catalyst of the processes as mentioned. To provide these systems with catalytic activity, aluminum is normally used in combination with cocatalyst. For example, known are methods of the polymerization, oligomerization and telomerization of olefins as well as alkylation of aromatic hydrocarbons with olefins which include metallic aluminum and organohalide /11/ /Patent U.S. Pat. No. 3,343,911. Nat. cl. 260-683.15.1969/.

The closest, as to technical essence and attainable result, to the method of the present invention for the preparation of the polyolefinic bases of synthetic oils is a method of oligomerization and polymerization of olefins under the action of a catalytic system including metallic aluminum and tetrachlorated carbon /2/ /I.C. USSR 803200, Nat. cl. BOI J 31/14. 1979/. A catalyst for the oligomerization and polymerization of olefins according to this method is produced by the interaction of the metallic aluminum and tetrachlorated carbon at temperatures of between 40 and 80° C. and an aluminum to tetrachlorated carbon weight ratio of I: (20-80) in the atmosphere of tetrachlorated carbon in absence of olefins in an inert atmosphere /12/ /I.C. USSR 803200. Nat. cl. BOI J 31/14. 1979/. In accordance with this method, at first, in the absence of olefins in the inert atmosphere there is obtained a solid coarse product of an uncertain composition to be further used as a catalyst of alpha-olefin oligomerization and isobutylene polymerization. We consider this method as a prototype for our method of the preparation of the polyolefinic bases of synthetic oils.

A disadvantage of the method-prototype is the use in this method, of tetrachlorated carbon within a catalytic system applied, with a high CCl₄/Al(O) ratio. This results in incorporating into the products a large amount (up to 3.0% w) of difficulty removable chlorine therefrom.

Another disadvantage of the method-prototype /12/ /I.C. USSR 803200. Nat. cl. BOI J 31/14. 1979/is low activity, low efficiency and low selectivity in terms of target products, of the catalytic system Al(O)—CCl₄ to be used according to the method per se.

A disadvantage of the method-prototype is also the multi-phase and high labour intensity of the preparation and use of a catalyst of olefin oligomerization and isobutylene polymerization from aluminum and CCl₄.

The general task of the present technical solution is the removal of all said disadvantages of conventional methods.

A basic concrete task of the present invention was elaboration of a method for preparing the polyolefinic bases of synthetic oils, with the use of a modified catalytic system for the cationic oligomerization of linear alpha-olefins (LAO) C₃-C₁₄, which would be characterized by enhanced activity and increased efficiency, would render possible controllability of oligomerization processes and, more importantly, would permit regulating the rate of oligomerization, increasing the yield of target low-molecular oligomer fractions (for example, dimers and trimers of decene-I), enhancing the ramified structure of a chain of oligomerization products and reducing solidification temperature thereof as well as would improve safety of its use in the process of olefin oligomerization. A second task of the present invention was simplification of the method of the preparation and use of the catalytic system of olefin oligomerization including metallic aluminum.

The tasks formulated in this invention are solved through the improvement of all the major stages of a method for preparing the polyolefinic bases of synthetic oils.

### DISCLOSURE OF THE INVENTION

A method of preparing the polyolefinic bases of synthetic oils, developed according to the present invention, just like any other similar method, comprises a step of conditioning an olefinic feedstock and solutions of the components of a cationic catalytic system, a step of isomerization of higher linear alpha-olefins, a step of oligomerization of olefinic feedstock under the action of a cationic aluminum-containing catalytic system, a step of releasing spent catalyst from an oligomerizate, a step of separating the oligomerizate into fractions and a step of hydrogenating the fractions released. Besides, after the step of oligomerization and/or after the step of releasing spent catalyst from the oligomerizate, the method further comprises a step of dechlorination of monochlorine-containing oligomers present in the oligomerizate and after the step of separation of the oligomerizate into fractions it comprises a step of depolymerization of high-molecular products released from the oligomerizate in the form of still residues in the step of separation of the oligomerizate into fractions (claim I as filed). These steps are intended for improving the technico-economic factors of the method, solving specific chemical problems and for enhancing the flexibility of the method worked out as to the products. In particular, the step of dechlorination of monochloroligodecenes, which form during oligomerization and are present in the oligomerizate, is designated for the conversion of the so-called “organic” chlorine, covalently attached to carbon in the monochloroligodecenes, to ion-attached with metals, the so-called “ion” chlorine. The latter obtained from chlorine-containing oligodecenes together with spent cationic catalyst, just like in the other methods of the cationic oligomerization of olefins or alkylation, is then removed from the oligomerizate by a method of water-alkali washing-off.

In accordance with the present invention, the polyolefinic bases of synthetic oils are prepared through the oligomerization of higher olefins in the mixtures of higher olefins to be oligomerized with the products of oligomerization thereof or in the mixtures of higher olefins to be oligomerized with the products of their oligomerization and aromatic hydrocarbons under the action of ternary, safe in transportation, storage and use, resistant in air, available cationic catalytic system Al(O)—HCl—(CH₃)₃CCl at temperatures between 110 and 180° C., Al(O) concentrations from 0.02 to 0.08 g-atom/l, HCl/Al(O) mole ratios variable in the range of 0.002 to 0.06 and RCl/Al(O) mole ratios variable in the range of 1.0 to 5.0, where in Al(O)— highly dispersed powdery aluminum with a particle size varying in the range of I to 100 mcm, for example Al(O), brand PA-1, PA-4, ACD-4, ACD-40, ACD-T (claim 2 as filed). The individual components of the system Al(O)—HCl—(CH₃)₃CCl are not the catalysts of higher olefin oligomerization. The precursors of cationic active centers and the cationic active centers of higher olefin oligomerization proper in this system are formed in a sequence of many chemical reactions between the components of the system. Metallic highly dispersed aluminum usable within the catalytic system under consideration consists of aluminum particles coated by a solid nonreactive alumina skin, owing to which fact it is stable in air and at temperatures between 20 and 110° C. is not actually reacted with HCl and (CH₃)₃ CCl. Al(O) reaction with HCl and (CH₃)₃ CCl begins only at temperatures exceeding 110° C. In accordance with the elaborated method of preparation of the polyolefinic bases of synthetic oils in the developed catalytic system, the aluminum is first reacted with hydrogen chloride. HCl at least partially destroys an aluminum oxide film on the surface of aluminum particles and provides a possibility of aluminum reaction proceeding with tert-butyl chloride. In other words, the hydrogen chloride in the system under consideration is a metallic aluminum activator. The reaction of aluminum with the tert-butyl chloride proceeds according to the following simplified diagram:

2Al(O)+3(CH₃)₃ CCl→2[(CH₃)₃C]_{1.5}AlCl_{1.5} (1).

The resultant sesquitert-butyl aluminum chloride (1), just like HCl, is reacted with the aluminum oxide skin on the surface of aluminum particles. This leads to accelerating a process of formation (1) and fully dissolving the metallic aluminum. Activation of aluminum is assured by an insignificant amount of hydrogen chloride to be dissolved in tert-butyl chloride (TBCh) in the process of its preparation by the reaction of isobutylene with the hydrogen chloride. A HCl/Al(O) mole ratio in the catalytic system Al(O)—HCl—(CH₃)₃CCl is varied in the range of from 0.002 to 0.06 by changing the concentration of HCl in TBCh from 0.015 to 0.5% w.

The concentration of aluminum in reaction media during the oligomerization of olefinic feedstock is varied in the range of 0.02 to 0.08 g-atom/l. With aluminum concentrations below 0.02 g-atom/l, oligomerization does not occur because of the inhibitory effects produced by impurities present in olefins and with aluminum concentrations above 0.08 g-atom/l, the specific consumption of catalyst components is markedly increased. The most favourable concentration of aluminum in reaction media is changed in the range of from 0.03 to 0.04 g-atom/l.

A TBCh/Al(O) mole ratio is varied in the range of from 1.0 to 5.0, the best mole ratio thereof is 3.5. With TBCh/Al(O) mole ratios below 3.5, only a part of metallic aluminum is dissolved, which is contained in the system Al(O)—HCl—(CH₃)₃CCl, and with TBCh/Al(O) mole ratios above 4.0, a chlorine content is sharply increased in oligomers. The primary cationic active centers in the catalytic system under consideration form according to the diagram:

[(CH₃)₃C]_{1.5}AlCl_{1.5}+(CH₃)₃CCl→(CH₃)₃C⁺{[(CH₃)₃C]_{1.5}AlCl_{2.5}}⁻ (2)

And TBCh performs the functions of cocatalyst.

Olefin oligomerization under the action of system Al(O)—HCl—(CH₃)₃CCl at high rate and high olefin conversion to products (above 95 mole %) proceeds at temperatures of from 110 to 180° C. During the oligomerization of alpha-olefins under the action of the system Al(O)—HCl—(CH₃)₃CCl there occurs the partial isomerization of alpha-olefins to a mixture of positional and geometric olefin isomers with the inner arrangement of double bonds which are cooligomerized with initial alpha-olefins. This results in the heightened degree of branching of the obtainable product molecules and a fall in solidification temperature thereof. In the case of oligomerization of decene-I, the use of the system Al(O)—HCl—(CH₃)₃CCl provides for a decrease in the portion of high-molecular oligodecenes C₆₀₊ from 50 (prototype) to 8% w. The oligodecenes formable under the action of this system contain from 4300 to 9970 ppm of chlorine (Table I). The use of the system Al(O)—HCl—(CH₃)₃CCl during oligomerization contributes to the solution of a problem of regulation of a fraction composition and ramified structure of the products of decene oligomerization. The consumption of components of this catalytic system in bulk does not exceed the corresponding indices of the best known (boron fluoride included) catalysts.

As a matter of fact, an important specific feature of the method elaborated is the fact that the interaction of aluminum with an activator (HCl) and a cocatalyst (TBCh) is carried out precisely in the process of oligomerization in the atmosphere of mixtures of olefins being oligomerized with oligomerization products and aromatic hydrocarbons added additionally (benzene, toluene, naphthalene). It is this solution that precludes work with concentrated highly reactive precursors and the reaction products of the formation of active centers and heightens safety of the method.

In accordance with the present invention, the higher oligomerized olefins accepted are represented by mixtures of linear or branched alpha-olefins with iso-olefins and olefins with the intramolecular arrangement of a double bond (with “inner” olefins) containing from 3 to 14 (predominantly, 10) carbon atoms, with the following ratio of ingredients, % w: alpha-olefins 0.5-99.0; iso-olefins 0.5-5.0; “inner” olefins—the balance up to 100% w. From data cited in Table 2, it is seen that, other conditions being equal, an increase in the number of carbon atoms, in the olefin molecules results in a reduced degree of branching of the oligo-olefin molecules and in an enhanced viscosity index thereof.

The most wide-spread methods are those for preparing the polyolefinic bases of synthetic oils wherein the olefinic feedstock used is represented by decene-I. This is necessitated by the fact that the trimers of decene-I released from the oligomers of decene-I, upon hydrogenation, are characterized by a unique complex of physical properties (kinematic viscosity at 100° C. is 3.9 cSt, viscosity index=130, solidification temperature=−60° C., flash point=215-220° C.). This combination of properties provides the possibility to use same as a base of widely used synthetic and semisynthetic motor (automobile, aviation, helicopter, tractor, tank) and other oils. Therefore, the best feedstock for the preparation of the polyolefinic bases of synthetic oils is decene-I. In accordance with the present method, the presence in initial decene-I, of iso-olefinic impurities (from 0.5 to 11.0% w) and olefins with the intramolecular arrangement of a double bond (from 0.5 to 5.0% w) does not actually affect the physico-chemical characteristics of the nonhydrogenated and hydrogenated fractions released from the products. In the course of cationic oligomerization, said decene-I is isomerized, before entering in an oligomeric product under the action of catalytic system Al(O)—HCl—(CH₃)₃CCl and other aluminum compound containing catalytic systems, to a mixture of positional and geometric decene isomers with the intramolecular arrangement of double bonds. The decenes with the intramolecular arrangement of double bonds (including individual decene-5) under the action of the system Al(O)—HCl—(CH₃)₃CCl are likewise easily (but more slowly) oligomerized as the decene-I. During the oligomerization of decenes with the intramolecular arrangement of double bonds there form more branched oligodecene molecules vs a decene-I instance (and, therefore, solidifying at lower temperatures).

From the classic stage mechanism of processes of cationic olefin oligomerization under the action of chlorine-containing (inclusive of organoaluminum) cationic catalysts there follows /1. J. Kennedy “Cationic Olefin Polymerization”. Moscow: MIR Publishers, 1978, 430 pages; 2. J. P. Kennedy. E. Marechal. Carbocationic Polymerization. N.-Y., 1982, 510 pages/ that the formable oligomers in these processes can contain “organically” bound chlorine (i.e. fixed with the carbon atom of oligodecene molecules within an oligomerizate). Theoretical calculations and experimental data go to show that from 2 to 10% of oligodecene molecules prepared under the action of usable cationic aluminum-containing catalysts contain one chlorine atom each and 90-98% of oligodecene molecules contain one double bond each. Upon deactivation and removal from the oligomerizate of spent cationic catalyst by a method of water-alkali washing-off, at +95° C., the oligomerizate contains from about 1000 to 10000 ppm (0.1-1.0% w) of chlorine linked with the carbon atoms of the oligodecene molecules. Chlorine gets into the oligodecenes with (actually irreversible) breaking of a chain as a result of capture by the growing carbocation of a chlorine anion from the anionic fragment of a cationic active center (AC). The diagram of this process as illustrated by the simplest catalyst RCl+AlCl₃(→R⁺AlCl₄⁻) is as follows (3):

R—(C₁₀H₂₀)ₙC₁₀H₂₀⁺AlCl⁻₄(AC)→R—(C₁₀H₂₀)ₙC₁₀H₂₀Cl+AlCl₃

Chlorine contained in an oligomerizate and target fractions causes the corrosion of equipment not only at all stages of a process for preparing oligodecenes but also in the process of using the oligodecene bases of synthetic oils. Because of this the chlorine should be removed not only from the main fractions of oligodecenes but also from the oligomerizate at the earliest stages of preparation thereof.

In accordance with the present invention, the dechlorination of monochlorine-containing oligomer molecules (RCI) present in an oligomerizate is carried out both after a stage of oligomerization and a stage of the release from the oligomerizate, of spent catalyst

Al(O)—HCl—(CH₃)₃CCl (claim I as filed). On elaboration of the present method for the preparation of the polyolefinic bases of synthetic oils, 5 variants of the solution of a dechlorination problem have been developed.

According to a first variant of the present method, RCl dechlorination is carried out by highly dispersed powdery metallic aluminum —Al(O) having a particle size varying in the range of from I to 100 mcm (for example, brands PA-I, PA-4, ACD-4, ACD-40, ACD-T) with Al(O)/RCl mole ratios varying in the range of from 0.5 to 2.0, in the temperature range of from 110 to 180° C. for 30 to 180 minutes (claim 4 as filed) (Table 3). Because of a high binding energy of C—Cl, chlorine from chloralkanes containing —CH₂Cl fragments is removed with the aid of chemical agents with great difficulty. Chlorine contained in R₃CCl fragments is most easily removed from the chloralkanes. Therefore, the test-indicator of rate and depth of the dechlorination of chloralkanes used was represented by I-chlorododecane containing the CH₂Cl fragment. From Table 3, it can really be seen that at temperatures not exceeding 95° C. under action of aluminum, brand PA-4, the dechlorination of I-chlorododecane and chlorine-containing oligodecenes does not take place. An increase in temperature to 120° C. or higher leads to the complete dechlorination of said chloralkanes. The reaction of dechlorination of 1-chlorododecane with aluminium in the atmosphere of decene-I results in almost 100% oligomerization of decene-I. This is indicative of the fact that the reaction of aluminum with I-chlorododecane proceeds in the same way as the reaction of aluminum with TBCh (i.e. with the intermediate formation of cationic active oligomerization centers). And the reaction of aluminum with RCl leads to the conversion of carbon covalently-attached chlorine to metal ion-attached chlorine which is removed from an oligomerizate at a stage of water-alkali washing off.

According to a second variant of dechlorination under the method sought for protection, monochlorine-containing oligodecenes (RCl) present in an oligomerizate are dechlorinated after a stage of oligomerization with triethyl aluminum (TEA) with TEA/RCl mole ratios varying in the range of from 0.5 to 2.0, in the temperature range of from 95 to 150° C. for 30 to 180 minutes (claim 5 as filed).

RCl dechlorination with triethyl aluminum under the aforesaid conditions proceeds according to the following diagram (4):

RCl+(C₂H₅)₃Al →{R⁺[(C₂H₅)₃AlCl]⁻}→RH+C₂H₄+(C₂H₅)₃AlCl (4)

From Tables 3, 4 it is seen that the dechlorination of I-chlorododecane with aluminum and triethyl aluminum occurs only at temperatures between 130 and 164° C. both in the absence of spent cationic catalyst and in the presence of products of its evolution during oligomerization. It is also evident that the conversion of decene-I to polymerization products through reaction of RCl dechlorination with aluminum and triethyl aluminum is increased. This is correlated with diagrams 3, 4, in accordance with which RCl reactions with the aluminum and triethyl aluminum proceed via a step of the formation of a cationic active center {R⁺[(C₂H₅)₃AlCl]⁻} which initiates the oligomerization of decene-I.

A common merit of the first and second variants of RCl dechlorination with the aid of Al(O) and TEA, according to the present invention, is the fact that RCl reactions with said dechlorinating agents are carried out right after oligomerization in the presence of spent, but not oligomerizate-released products, of the conversion of a usable cationic catalytic system Al(O)—HCl-TBCh. The alkylaluminum chlorides resulting from dechlorination are released from the oligomerizate simultaneously with spent catalyst in a step of water-alkali washing-off. This simplifies the technological execution of this step but calls for an increased consumption of dispersed aluminum or use of TEA.

Primary and secondary chloralkanes under normal conditions (i.e. at temperatures not exceeding 100° C.) are not hydrolyzed with water and aqueous solutions of sodium or potassium hydroxides. In accordance with the a third variant of the present method for preparing the polyolefinic bases of synthetic oils, the dechlorination of monochlorine-containing oligomers (RCl), which form during oligomerization and are present in an oligomerizate is carried out before or after the step of releasing spent catalyst with an alcohol (butynol or hexanol —ROH) solution of a potassium or sodium hydroxide (MOH) with MOH/RCl mole ratios varying in the range of from 1.1 to 2.0, in the temperature range of from 120 to 160° C. for 30 to 240 minutes (Table 5) (claim 6 as filed). Instead of said ROHs, the KOH solvent that might be used is represented by a monoethyl ethylene glycol ether. MOH concentration in ROHs is varied in the range of from 1 to 5% w. From Table 5 it is seen that, other conditions being equal, the rate of reaction and dechlorination conversion markedly decrease while replacing KOH with NaOH. For this very reason, KOH is preferable. The reaction of RCl dechlorination with alcohol MOH solutions proceeds slowly even at 120° C. A rise in the temperature from 120 to 150° C. results in a sharp increase of the speed of reaction. At 150° C. the reaction is completed in 60 minutes. RCl dechlorination according to this variant proceeds according to a diagram including the intermediate formation of alkali metal alkoxides which are further reacted with RCl chloralkanes: KOH+C₄H₉OH→C₄H₉OK+H₂O; C₄H₉OK+RCl→KCl+C₄H₉OR

Sodium and potassium chlorides in said alcohols under chloralkanes dechlorination reaction conditions are insoluble, which permits separating same from reaction mass by precipitation followed by an MCl salt precipitate dissolved with water.

The simplest variant of those developed for dechlorinating chlorine-containing oligodecenes resides in that the dechlorination of monochlorine-containing oligomers (RCl) present in an oligomerizate is carried out after a step of releasing spent catalyst by way of thermal RCl dehydrochlorination in the temperature range of from 280° C. to 350° C. and at a pressure between I and 2 bar for 30 to 180 minutes by blowing a hydrogen chloride to be released with nitrogen, carbon dioxide, methane (natural gas) or overheated water vapor (Table 6) (claim 7 as filed). The thermal dehydrochlorination of this alternative embodiment is carried out in the preheater-vaporizer of an atmospheric column under a vigorous stirring of the oligomerizate by a blowing gas or overheated water vapor. This variant of dechlorination of chlorine-containing decene oligomers has both merits and defects: it provides the deep degree of dehydrochlorination of the oligomerizate (97-98%), does not call for using new agents but leads to the complication of execution of the atmospheric column.

The thermal dehydrochlorination of chlorine-containing compounds starts off with temperatures exceeding 250° C. The speed of thermal dehydrochlorination considerably depends on the structure of a chlorine-containing compound. In the case of chlorine-containing compounds having bonds beta-C—H in relation to bonds C—Cl, the most thermally stable are primary alkyl halides, the least thermally stable are tertiary alkyl halides. The thermal R₃CCl dehydrochlorination proceeds at marked rate even at 100° C. From the makeup of initial agents and stage mechanism of decene-I oligomerization there follows that an oligomerizate can contain all theoretically possible types of chlorine-containing compounds for each particular case (primary, secondary and tertiary alkyl halides comprising and not comprising bonds beta-C—H). The rates and, other conditions being equal, degrees of dehydrochlorination of the primary, secondary and tertiary alkyl halides comprising the bonds beta-C—H increase more often than not as the temperature rises.

With temperatures of between 250 and 300° C., the dehydrochlorination of a chlorine-containing oligomerizate occurs relatively slowly and apparently is reversible during a period of 6-10 hours. Hydrogen chloride released in dehydrochlorination events can at once be attached to the oligodecene molecules comprising double bonds. This is facilitated by a relatively high concentration of the double bonds of different types in the oligodecene molecules.

Other static conditions being equal, the rate and degree of dehydrochlorination of an oligomerizate increase as the temperature rises from 300 to 330° C. The depolymerization of oligodecenes does not take place thereat. With the temperature of 330° C. and the residence time of two hours almost all of the organically bound chlorine contained in oligomers is removed from the oligomerizate in the form of HCl. Upon thermal treatment, in the oligomerizate there remains about 20 ppm of organically bound chlorine (i.e. about 0.002% w). Under the conditions of blowing hydrogen chloride and vaporous components from the oligomerizate with nitrogen at 330° C. and the residence time of one hour in the oligomerizate only 3 ppm of the organically bound chlorine remain. From data available it follows that the degree of removal of chlorine-containing hydrocarbons from the oligomerizate=99.94% w. The content of organo-chlorine compounds in a recycled decene-decane fraction does not exceed 160 ppm (which corresponds to 0.016% w). This fraction can be mixed with fresh decene being admitted to an installation and the resulting mixture can be used as feedstock for the preparation of oligodecenes.

The thermal dehydrochlorination of chlorine-containing oligodecene molecules at the temperatures of between 300 and 330° C. and with the total residence time of two hours proceeds almost quantitatively according to the following diagram: R—(C₁₀H₂₀)ₓ₋₁—C₁₀H₂₁Cl→HCl+R—(C₁₀H₂₀)ₓ₋₁—C₁₀H₂₀(═).

The released hydrogen chloride is blown with nitrogen or overheated water vapor and is admitted together with water and hydrocarbon vapors first to a condenser and therefrom to a scrubber for HCl neutralization with an aqueous sodium hydroxide solution.

To prevent the liberation of hydrogen chloride in free state into a vapor-gas phase, the dehydrochlorination of monochlorine-containing oligomers (RCl) present in an oligomerizate, according to the present invention (claim 8 as filed) is carried out in the temperature range of from 300 to 330° C. in the presence of dry alkali metal hydroxides (MOH) with MOH/RCl mole ratios varying from 1.1 to 2.0 (Table 7). To provide the maximum possible degree of dispersion of particles of oligomerizate insoluble dry alkali metal hydroxides they are obtained in accordance with the present invention (claim 9 as filed) directly in the oligomerizate through the distillation of water while heating the mixture of a spent catalyst-free oligomerizate and a 5-40% aqueous solution of an alkali metal hydroxide at temperatures varying in the range of from 100 to 200° C. (Table 7). With a temperature rise from 100 to 200° C. there take place evaporation and complete removal of water from the reaction mass. The subsequent dehydrochlorination of chlorine-containing oligodecenes is carried out for one hour at the temperatures of 300, 310, 320 and 330° C. From Table 7 it is seen that, other conditions being equal, the residual content of chlorine in an oligomer, as the temperature rises from 300 to 330° C., is sharply reduced to achieve 8 ppm at 330° C., with the degree of dehydrochlorination of 95.56% w. The hydrogen chloride does not come out into a gas phase—it is fully reacted with a potassium hydroxide and is found as KCl in a solid precipitate to be dissolved in water in full. Washing of the dehydrochlorinated oligomerizate with water, upon its discharge from a reactor, and the analysis of wash water for chlorine go to show that some portion (less than 0.5% w) of the formable potassium chloride is kept in suspension in the dehydrochlorinated oligomer.

The elaborated variants of dechlorination of the chlorine-containing oligodecene molecules (i.e. chloralkanes) are universal and can be used independently in solving similar tasks in other chemical processes, oil refining as well as in the dechlorination of mono-, di- and polychlorine-containing aliphatic and aromatic hydrocarbons, oligomers, polymers, oil fractions and diverse liquid and solid chlorine-containing organic waste products (claim 10 as filed).

By way of example, Table 8 shows data on dehydrochlorination of liquid polychloroparaffins containing 44% w chlorine with a butanol KOH solution. The solution of this problem is particularly made use of in obtaining synthetic boiled oil and acetylene oligomers.

The method of preparing the polyolefinic bases of synthetic oils, as sought for protection, provides for the utilization of high-molecular oligodecene fractions, which find no application, through depolymerization thereof. A step of depolymerization of high-molecular oligodecenes is intended for correcting the molecular-weight distribution and fractional composition of oligodecenes to be obtained in a step of oligomerization. The depolymerization of high-molecular products released as still bottoms in a step of oligomerizate separation into fractions, according to the method developed (claim 11 as filed) is carried out by heating same at the temperatures of from 330 to 360° C. and pressures of from 1.0 to 10.0 mm Hg for 30 to 120 minutes, with the continuous removal of products from a reactor of depolymerization to a system of atmospheric and two vacuum columns to separate the oligomerizate into fractions.

The separation of a decene oligomerizate into fractions and depolymerization of separated still bottoms comprising high-molecular oligodecenes were conducted on a vacuum-pumping assembly of French company “GECIL”. The usable computerized automatic assembly (model “minidist C”) comprises two columns, two electric heated stills, 10 and 22 liters in volume, a glass collector, about 10 receptacles for the separated fractions, a vacuum station, a vacuum pump, a vacuum gage and two apparatuses for measuring the temperature in the still and a column head. The collector was equipped with the automatic control system of fraction collection rate. The vacuum system of this installation renders possible fractionation of an oligomerizate with strictly specified residual pressure. A first column with regular grid packing and irrigation can function under atmospheric or reduced pressure (up to 2.0 mm Hg). A second column-vacuum column (without packing and irrigation) can function under high vacuum (even when a residual pressure is equal to 1.0-0.01 mm Hg) at temperatures up to 370° C. The distillation installation under consideration is equipped with also an automatic unit for determining fraction mass. All information about the particulars of separation of the oligomerizate enters a computer to be accumulated and processed therein. In particular, the computer specifies the virtual temperature of a process under atmospheric pressure (Tᵥ), according to a special program, on the basis of the concrete temperature values of the still and column's head and residual pressure in the column, which coincides with the nominal temperature of fractionation (Tₙ) to be found with the aid of a conventional monographic chart T-P. The separation of the oligomerizate into fractions was carried out on the first column with regular packing (15 theoretical plates) with a still of about 20 liters. The depolymerization of high-molecular oligodecenes was carried out on the second (vacuum) column without the regular packing and irrigation with a still of about 10 liters.

The electric heated still of the first column was loaded with 5 to 10 kg of a catalyst and organically bound chlorine-free oligomerizate.

The separation of an oligomerizate into fractions was carried out under the conditions of a slow temperature rise from 20 to 300° C. Low-boiling components and also PAO-2 and PAO-4 from the oligomerizate were separated out the first column with packing and irrigation. PAO-6 and PAO-8 were separated out on a second vacuum system.

It is has been found that during the separation of a decene oligomerizate into narrow fractions at temperatures exceeding 330° C. there occurs thermal oligodecene depolymerization. In the range of temperatures of between 300 and 330° C., at a residual pressure of below 5 mm Hg, the decene trimers and tetramers left over therein are separated out of the oligomerizate. In the still there occurs substantially full depolymerization of high-molecular decene oligomers at 360° C. for three hours, at a residual pressure in a column of less than 3 mm Hg. And the resultant products were divided into fractions. It has been found that the lightest fraction separated at temperatures of 20 to 150° C. out of the depolymerization products of high-molecular oligodecenes consists of a mixture of olefins (predominantly decenes) with vinyl (53.6%), trans-vinylene (18.1%) and vinylidene (28.3%) double bonds. The products separated out in the temperature range of 150 to 240° C. and 240 to 300° C. are decene dimers and trimers, respectively. This is confirmed by a method of gas chromatography and a coincidence of the main properties of these fractions with the properties of standard samples of the decene dimers and trimers. The still bottoms contained, upon separation of break-down products into fractions, the nondepolymerized high-molecular decenes oligomers.

The composition of products described goes to show that during the thermal treatment of high-molecular oligodecenes there occurs (probably statistical) depolymerization thereof. The effect revealed is of substantive practical significance as rendering possible the reprocessing of, if need be, high-molecular oligodecenes into di-, tri- and tetramers of decenes by a simple method. The results of separation of a decene oligomerizate into fractions under the conditions of high-molecular component partial depolymerization are given in Table 9.

Depolymerization of decene high-molecular oligomers (PAO-10÷PAO-20) with kinematic viscosity at 100° C. was purposefully carried out at temperatures of 330 to 360° C., with a residual pressure on the column's top of a depolymerizer of I mm Hg while in the column's still—3-5 mm Hg.

To grasp a clear idea of the work of a depolymerizer, given below is a description of one of the typical experiments. Example. In the still of a depolymerizer were loaded 3000 g of a decene oligomer having a boiling temperature of above 360° C. at a residual pressure on the top to a column of 1 mm Hg. As a result of depolymerization of this oligodecene at 350° C. for three hours, 2258 g (75.27% w) of depolymerization products were distilled off. Oligodecene-I depolymerization products separated through the top of a reactor-column in the total amount of 2258 were divided again into the following fractions:

| | |
|---|---|
| Fraction boiling out | Fraction, yield |

| Nature of fraction | Interval T, ° C. | g | % w |
|---|---|---|---|
| Gaseous HC | 20-30 | 60 | 2.6 |
| Liquid HC | 30-150 | 154 | 6.8 |
| PAO-2 | 150-240 | 156 | 6.9 |
| PAO-4 | 240-330 | 830 | 36.8 |
| PAO-6 | 330-360 | 1058 | 46.9 |

On depolymerization of oligodecenes at a boiling temperature of above 300° C., at the temperature of a still of 360° C. and of the walls of a depolymerizer 350° C., at a residual pressure in a condenser of I mm Hg the following results have been obtained:

| | Interval T, | Fraction, yield | |
|---|---|---|---|
| Fraction nature | fraction boiling out, ° C. | g | % w |
| Gaseous HC | 20-30 | 62.8 | 2.0 |
| Liquid HC | 30-150 | 188.4 | 6.0 |
| PAO-2 | 150-240 | 376.8 | 12 |
| PAO-4 | 240-330 | 471-0 | 15.0 |
| PAO-6 | 330-360 | 2041.0 | 65.0 |
| | | Σ = 3148.0 | 100.0 |

Note:
HC—hydrocarbons; T—temperature.

For the best conditions of functioning of a depolymerizer to be revealed, a study has been given to the influence of temperature on the kinetics of the thermal depolymerization of high-molecular oligodecene with a boiling temperature of above 330° C.

From data obtained there follows:

1. A depolymerizer achieves steady-state temperature conditions for 10-20 minutes.

2. With attainment of the specified temperature conditions, the thermal depolymerization of high-molecular oligodecene proceeds at uniform rate.

3. The rate of depolymerization, yield of depolymerization products and conversion of high-molecular oligodecene to target products monotonically increase as the temperature rises from 340 to 360° C.

4. The observable activation energy of depolymerization equals 27.5 kcal/mol, 1 g A=9.9073.

5. At 360° C. the conversion of high-molecular oligodecene to depolymerization products amounts to 70% or more, which are distilled off from a depolymerizer at said temperature and a residual pressure in a still of not more than 3 mm Hg to be directed to a column for repetitive separation.

6. Depolymerization products differ from those of decene-I oligomerization not only in that alongside the molecules having inner (vinylene) and vinylidene double bonds they contain a considerable number (about 30 mole %) of molecules with vinyl double bonds but also in physico-chemical properties (Table 9, 10).

7. Thermal depolymerization of high molecular oligoolefins represents a chemical endothermal process. To provide the depolymerization of 1000 kg of high molecular oligodecenes in one hour, provision should be made of a heater with the total capacity of 116 kWt.

The heat balance of a depolymerizer is as follows:

Heat supply—116 kWt/hr. Heat consumption:
heating of high-molecular oligodecenes up to 360° C.—30 kWt/hr;
endothermal reaction of depolymerization at 360° C.—33 kWt/hr;
evaporation of the resulting products—28 kWt/hr;
heat reserve—11 kWt/hr;
heat loss 14 kWt/hr.

Be it noted that process conditions (temperature, pressure) produce a marked effect on the composition of depolymerization products and characteristics thereof (Tables 9, 10). A particular great effect is produced on the composition and characteristics of depolymerization products by the rate of their removal from a reactor. As temperature and pressure grow and the time for distilling the products off from a depolymerizer is reduced, the intensity of oligodecene depolymerization is markedly increased.

The type of chromatograms of dimers and trimers and also the high values of solidification temperatures thereof show that during the thermal depolymerization of high-molecular oligodecenes at a high residual pressure of over 10 mm Hg of a regular packed column with irrigation there occurs paraffinization thereof. Said thermal depolymerization conditions when the products of primary depolymerization are not removed from a reaction zone, but repeatedly recycled to a still are probably conductive to the chain break-down of oligodecenes. This conclusion is illustrated by an example which was realized on a column without regular packing and irrigation at a residual pressure of between 1.0 and 0.6 mm Hg. In this example, the initial depolymerizate used was represented by still bottoms separated out of a decene depolymerizate upon water-alkali dehydrochlorination. It contained about 30 ppm of chlorine and had the following composition:

| Retention time, min. | Component | Content, % w |
|---|---|---|
| 10.9 | Trimer | 19.93 |
| 13.8 | Tetramer | 32.91 |
| 15.9 | Pentamer | 21.75 |
| 17.7 | Hexamer | 11.48 |
| 24.2 | Heptamer | 13.93 |

The still of a depolymerizer-column was loaded with 1484.6 g of the cited dehydrochlorinated high-molecular oligodecene. At first the oligomerizate was gradually heated to 360° C. in the still under stirring by a powerful electromagnetic stirrer. The depolymerization of high-molecular oligodecene started off with the temperature of 330° C. and was carried out generally at 360° C. The real and virtual temperature of the still and the column's head were determined by heat loss, heat consumption for warming up the oligomerizate and depolymerization products, for the thermal depolymerization of oligodecenes and for evaporation of depolymerization products. On a thermogram, the course of all these endothermic processes was manifest in the form of several endo-effects. Rate of depolymerization (rate of distillation of thermal depolymerization products, to be more exact) changed in time in a complicated manner. At first, a process rate monotonically increased to about 13 ml of products per minute, as the temperature was raised from 330 to 360° C. But on the 170^{th} minute the abrupt increase (almost 3 times) in the rate of distillation of thermal depolymerization products occurred. Inasmuch as the temperature and residual pressure remained invariable thereat, one can surmise that said acceleration of the process is defined by its degenerate chain character.

As a result of depolymerization of high-molecular oligodecenes under the above-mentioned conditions, a trap accumulated a condensate, two fractions of products were separated out and still bottoms were obtained. The condensate consisted of primarily (98.3% w) hydrocarbons C₄-C₁₂.

A portion of hydrocarbons (paraffins, olefins) in a condensate monotonically diminished from C₄ to C₁₂. This composition of the condensate probably reflects the relationship of different branchings in the oligodecene molecules. If this assumption corresponds to the facts, one can make a conclusion that the oligodecene molecules mostly contain butyl branchings. A first fraction of products of thermal depolymerization separated in an amount of 657.7 g, with the virtual temperatures of from 209 to 575° C. has a highly complicated composition:

| Retention time | Component | Content, % w |
|---|---|---|
| 1.09 | C₄-C₁₂ | 0.34 |
| 3.83 | C₁₂-C₁₈ | 1.09 |
| 7.33 | Dimer | 2.39 |
| 10.81 | Trimer | 3.42 |
| 13.71 | Tetramer | 3.88 |
| 15.93 | Pentamer | 21.42 |
| 17.75 | Hexamer | 46.96 |
| 25.20 | Heptamer | 20.49 |

As seen from the above Table, this fraction is a mixture of 1.43% w of the low-molecular products of thermal depolymerization and also initial and depolymerization resultant di-, tri-, tetra- and more high-molecular oligodecenes. The solidification temperature of this fraction −39° C. It drops to −49° C. upon separation therefrom of low molecular C₄-C₁₈ hydrocarbons.

A second fraction of thermal depolymerization products of high molecular oligodecenes separated in an amount of 295.1 g, with virtual temperatures of 575-534° C. (at a residual pressure of 0.6 mm Hg), just like the first fraction, is a complex mixture of the low-molecular products of thermal depolymerization and also initial and depolymerization resultant di-, tri-, tetra- and more high-molecular oligodecenes. The solidification temperature of this fraction −33° C., but is reduced to −45° C. upon separation therefrom of low-molecular C₄-C₁₈ hydrocarbons. The content of low-molecular depolymerization products (3.0% w) in the second fraction is over two times as high as the content of low-molecular depolymerization products (1.43% w) within the first fraction. This is indicative of an increase in the depth of depolymerization. The total conversion of initial oligodecenes to depolymerization products exceeded 70% w. The total output of the second and third fractions (952.8 g)=64.18% w.

Still residues (458.8 g=30.9% w) consist of a mixture of initial and depolymerized high-molecular oligodecenes:

| Retention time | Component | Content, % w |
|---|---|---|
| 16.10 | Pentamer | 0.45 |
| 17.98 | Hexamer | 2.43 |
| 25.41 | Heptamer+ | 97.11 |

It has the solidification temperature −30° C.

A combination of data obtained allows the drawing of general conclusions concerning the following:
high-molecular oligodecenes using a method of thermal depolymerization, at temperatures of 330-360° C. and a residual pressure of 1.0 mm Hg can be reprocessed with high conversion to a mixture of low-molecular products;
from a mixture of the thermal depolymerization of high-molecular products, using a method of vacuum distillation, fractions of products can be separated as to fractional composition and viscosity properties approximating PAO-2, PAO-4, and PAO-6;
solidification temperatures of the separated fractions exceed those of PAO-2, PAO-4 and PAO-6 separated out of an initial oligomerizate, albeit are significantly below the solidification temperatures of the corresponding, as to viscosity properties, noncompounded fractions of mineral oils.

It case of necessity, this permits recommending them, upon hydrogenation, to be used in mixture with main relevant products as the bases of synthetic and semisynthetic oils. Provision of a step of depolymerization renders possible treatment of all restricted consumable high-molecular oligodecenes with a kinematic viscosity at 100° C. being variable from 10 to 20 cSt to widely used polyolefins with a kinematic viscosity at 100° C. being variable from 2 to 8 cSt (i.e. PAO-2, PAO-4, PAO-6 and PAO-8, respectively).

Separation of an oligomerizate into narrow fractions according to the method of patent protection sought, as already stated, is carried out in the way similar to other conventional methods except for the fact that the high vacuum in separation columns is assured by an original vacuum vapor-jet system.

Products separated in a step of division of an oligomerizate into fractions represent in all cases, the mixtures of unsaturated and chlorine-containing oligodecene molecules. The residual content of chlorine in separable fractions does not exceed 10 ppm. To increase the oxidation of stability of the obtainable products in all conventional methods, they are hydrogenated.

Hydrogenation of oligomerizate-separated narrow oligo-olefin fractions according to the present invention, is carried out under the action of a palladium alumina-supported catalyst (predominantly—Pd(0.2% w)/Al₂O₃) modified with anhydrous NaOH taken in an amount of 30 to 100% w, as calculated for hydrogenation catalyst, at temperatures varying in the range of from 200 to 250° C. at a hydrogen pressure of 20 at.

The temperature and pressure at the stage of hydrogenation according to the method sought for protection are appreciably lower than in the case of other known methods. The hydrogenation of oligodecene narrow fractions under the aforesaid conditions makes possible the hydrogenation of not only C═C but also the bonds C—Cl of the oligodecenes. The hydrogenation of oligodecene fractions in the presence of anhydrous NaOH facilitates the enhanced activity and efficiency of hydrogenation catalyst as a result of neutralization of hydrogen chloride resulting from the hydrogenation of bonds C—Cl left over in the hydrogenated fractions of chlorine-containing oligodecenes. Besides, this solution removes the corrosion of hydrogenation reactors and auxiliary equipment and prevents accelerated deactivation with the hydrogen chloride, of palladium hydrogenation catalyst.

On development of a method for the preparation of the polyolefinic bases of synthetic oils, use was made of the following reagents: decene-I separated out of the products of ethylene oligomerization under the action of triethyl aluminum at OAO “Nizhnekamskneftekhim” (Specifications 2411-057-05766801-96) and also decene-I separated out of the products of ethylene oligomerization under the action of triethyl aluminum in Neratovize town (Czechia), company “Spolana”. The usable decene-I manufactured by OAO “NKNX” had the following group composition: CH₂═CH— 83.4 mole %; trans-CH═CH— 5.44 mole %; CH₂═C<11.2 mole %. Before use, the decene-I was dried over calcined molecular sieves NaX at 600° C.; n-heptane of “standard” brand used as solvent for preparing the solutions of catalyst components was dried through distillation over sodium wire; AOC of the general formula RₙAlCl₃₋ₙ, (R—C₂H₅; n=1.5, 3.0) was purified by distillation at reduced pressure. The conditioned olefins, solvents and AOC were kept in an inert atmosphere in hermetically sealed vessels. AOC were used as diluted n-heptane solutions.

Decene-I oligomerization was carried out under the action of systems Al(O)—HCl—(CH₃)₃ CCl (TBCh) and Al(O)—(C₂H₅)_{1.5}AlCl₁₋₅ (EASX)—(CH₃)₃ CCl, in which HCl and EASX performed the functions of aluminum activators, ACD-4, ACD-40, PA-I and PA-4 brands. On elaboration of a method of patent protection sought, use was mainly made of PA-4 aluminum. The rate of decene-I oligomerization under the action of said catalytic systems is limited by the rate of dissolving the aluminum by tert-butyl chloride. This process precedes the formation of active centers of decene-I oligomerization. Inclusion in said catalytic systems, of aluminum activators reduces or eliminates an induction period.

In both said systems use was made of, a cocatalysts, tert-butyl chloride —(CH₃)₃CCl which was obtained by the reaction of isobutylene with dry HCl. TBCh contained 0.5% w HCl (Specifications Jun. 9, 2007-1338-83).

In preliminary investigations it was found that the best aluminum activator, as to a combination of characteristics, is HCl and the best cocatalyst in the process of decene-I oligomerization is TBCh. Therefore, main investigations were carried out with the use of a catalytic system Al(O), PA-4-HCl-TBCh brand.

Decene-I oligomerization was carried out in a thermostatically controlled dried glass or metal mixing reactor in the atmosphere of dry argon under a continuous vigorous stirring of reaction mass with the aid of an electromagnetic stirrer. Testing of catalytic systems during decene-I cationic oligomerization and other olefins was conducted in the following manner: in a reactor controlled thermostatically to a specified temperature was gradually loaded with aluminum, decene-I or other olefin and then a HCl solution in TBCh. Al was loaded into the reactor in the form of a pre-conditioned inert-atmosphere charge stored in a glass soldered ampule. Following loading in the reactor, all said agents (usually immediately) olefin oligomerization began to be accompanied by a noticeable increase in the temperature of reaction media (oligomerizate). Reaction was continued for a suitable time and then stopped suddenly by introducing into the reactor, an aqueous alkaline solution (NaOH) or ethanol. The prepared oligomerizate was further unloaded from the reactor into an intermediate container. The spent (deactivated) catalyst from the oligomerizate was removed by a method of water-alkali and water washing-off.

The decene-I content (or another initial olefin) in reaction mass (oligomerizate) in the course and after oligomerization (i.e. current and overall conversion) was determined by a method of gas chromatography on instruments LXM-8-M, LXM-2000 and “Hewlett-Packard 588OA” (inner standard-pentadecane) and by a method of IR-spectroscopy on an instrument “Specord M-80”, for which purpose at the specified moment a portion of the reaction mass was taken away from an argon-atmosphere reactor which was at once mixed with ethanol or a 5% NaOH aqueous solution under vigorous stirring conditions. The reaction mass was repeatedly washed, upon termination of oligomerization, with distilled water in a closed funnel.

The unreacted decene-I and also di-, tri- and tetradecenes from an oligomerizate were separated on a vacuum column with a still electrical heated to 360° C. at a residual pressure of from I to 10 mmHg.

The fractional composition of an oligomerizate was determined on chromatographs LXM-8-MD, LXM-2000 and “Hewlett-Packard 588OA” with ionization flame detectors under temperature programming conditions of from 20 to 350° C. at the rate of a temperature rise of 8-10 deg/min. At the time of chromatographing olicodecenes, use was made of stainless steel columns (0.4×70-0.4×200 cm) filled with chromaton NAWDMCS with 3.0% silicon OV-17, chromosorb W-AW with 3% Dexil-300 or Paropak-Q. The average equivalent diameter of particles of said carriers is 0.200 to 0.250 mm. The rate of feed of a pre-purified carrier gas (helium, nitrogen) was ˜40, hydrogen ˜30, air ˜300 ml/min. A sample in a chromatograph-evaporator was introduced using a microsyringe (0.2-1.0 mcl) upon achievement of the evaporator's temperature of 350° C. Assay duration—50 minutes. Chromatographic peaks were identified by a method of adding reference-point hydrocarbons (pentadecane) and by way of comparing with chromatograms of hydrocarbon mixtures of a known composition. The quantitative processing of chromatograms was carried out according to integral peak areas which were determined using a computer integrator or by a method of triangulation. The fractional composition of the oligomerizate was likewise determined by a method of fractionation thereof on a vacuum rectifying column. The oligomerizate was likewise determined by a method of fractionation thereof on a vacuum rectifying column. The results of these quantitative assays coincided with an accuracy of ±3% w.

The unsaturated state of oligomers (i.e. the content in the oligomer molecules, of double bonds) was determined by a method of ozonolysis on a double bond analyzer ADC-4M.

The structure of nonconverted decenes and oligomerization products was quantitatively determined by an IR-spectroscopy method on an instrument “Specord M-80” and also methods of PMR and NMR¹³C spectroscopy. PMR and NMR¹³C spectra were registered at room temperature on a pulse spectrometer NMR AC-200P (200 MHz, firm “Bruker”. For plotting of PMR and NMR¹³C spectra, 10-20% solutions of products in deuterochloroform were prepared. Tetramethylsilane was used as a standard.

Ion chlorine impurities in oligomers were determined by the Folgard argentometric method by titrating an aqueous extract. Chlorine covalently attached to the oligomer molecules was first converted to an ion form by way of wet burning a sample in a crystal reactor or with the aid of sodium biphenyl according to a UOP 395-66 method followed by argentometric titration according to Folgard. In some cases, the content of an oligomer molecule covalently attached chlorine was determined by a roentgenfluorescent method on a spectrometer “SPECTRO XEPOS” about a calibration curve.

To grasp a clear idea of the present invention, illustrations are afforded (Tables 1-10) of realization of a method of the invention for obtaining the polyolefinic bases of synthetic oils. These examples demonstrate but do not exhaust the possibilities of the invention.

A combination of solutions with reference to different aspects of the invention, as described in the specification, allows one to affirm that developed is a new method for the preparation of the polyolefinic bases of synthetic oils. The method per se comprises the steps of conditioning olefinic feedstock, preparing and dosing in a reactor, the solutions and suspension of components of a catalytic system Al(O)—HCl-TBCh, alpha-olefins isomerization and higher olefins oligomerization and mixtures thereof under the action of the catalytic system Al(O)—HCl-TBCh, separation of spent catalyst, division of an oligomerizate into fractions and hydrogenation of the fractions separated under the action of catalyst Pd (0.2% w)/Al₂O₃+NaOH. The invention contributes to improvement of all steps of the method elaborated.

To remove the corrosive activity of products, the method further comprises a step of dechlorination of the chlorine-containing oligo-olefins present in an oligomerizate with metallic aluminum, triethyl aluminum, alcohol KOH solutions or thermal dehydrochlorination of chlorine-containing polyolefins in the absence or presence of KOH. For technico-economic method factors to be improved by increasing an output of polyolefin target fractions, with a kinematic viscosity of 2-8 cSt at 100° C., the method further comprises a step of the thermal depolymerization of restricted consumable high-molecular polyolefins with a kinematic viscosity of 10-20 cSt at 100° C. to target polyolefins with a kinematic viscosity of 2-8 cSt at 100° C.

TABLE 1
Effect of diverse factors on composition and structure of decene-1
oligomerization products under action of system Al(0), brand
PA-4-HCl—(CH₃)₃CCl (TBCh). Decene-1 = 20 ml.

| | | | | HCl | | | | | |
|---|---|---|---|---|---|---|---|---|---|
| Example | Aluminum | | | TBCh, | | TBCh | RCl | | τ, |
| N^{o} | g | mole | mole/l | % w | mole | mole/l | Al | T, ° C. | min. |
| 1 | 0.036 | 0.00133 | 0.0665 | 0.5 | 0.00665 | 0.3325 | 5 | 100 | 60 |
| 2 | 0.036 | 0.00133 | 0.0665 | 0.5 | 0.00665 | 0.3325 | 5 | 110 | 120 |
| 3 | 0.036 | 0.00133 | 0.0665 | 0.5 | 0.00665 | 0.3325 | 5 | 110 | 60 |
| | | | | | | | | | 120 |
| 4 | 0.036 | 0.00133 | 0.0665 | 0.5 | 0.00665 | 0.3325 | 5 | 120 | 60 |
| 5 | 0.0144 | 0.0005 | 0.026 | 0.5 | 0.00266 | 0.133 | 5 | 110 | 60 |
| | | | | | | | | | 120 |
| 6 | 0.0216 | 0.0008 | 0.04 | 0.5 | 0.004 | 0.2 | 5 | 110 | 60 |
| | | | | | | | | | 120 |
| 7 | 0.0288 | 0.0010 | 0.0533 | 0.5 | 0.0053 | 0.265 | 5 | 110 | 60 |
| 8 | 0.036 | 0.00133 | 0.0665 | 0.5 | 0.00665 | 0.3325 | 5 | 110 | 120 |
| 9 | 0.036 | 0.00133 | 0.0665 | 0.5 | 0.00399 | 0.1995 | 3 | 110 | 60 |
| | | | | | | | | | 120 |
| 10 | 0.036 | 0.00133 | 0.0665 | 0.5 | 0.00532 | 0.266 | 4 | 110 | 60 |
| 11 | 0.036 | 0.00133 | 0.0665 | 0.5 | 0.00665 | 0.3325 | 5 | 110 | 120 |
| 12 | 0.036 | 0.00133 | 0.0665 | 0.5 | 0.00665 | 0.3325 | 5 | 110 | 120 |
| 13 | 0.036 | 0.00133 | 0.0665 | 0.17 | 0.00665 | 0.3325 | 5 | 130 | 60 |
| 14 | 0.036 | 0.00133 | 0.0665 | 0.015 | 0.00665 | 0.3325 | 5 | 150 | 60 |

| | | R/1000CH₂ | | | | | Tetra- | |
|---|---|---|---|---|---|---|---|---|
| Example | S, | | trans- | Chlorine, | Dimer, | Trimer, | mer, | Pentamer + |
| N^{o} | % w | CH₃ | CH═CH | ppm | % w | % w | % w | % w |
| 1 | 100 | 338.9 | 15.7 | 7618 | 38.44 | 22.4 | 11.9 | 2.63 |
| 2 | 98.9 | 451.2 | 12.1 | | 37.7 | 32.15 | 11.1 | 1.9 |
| 3 | 100 | 451.3 | 15.2 | | 38.57 | 30.9 | 10.0 | 1.9 |
| | 100 | 461.9 | 12.6 | 7214 | | | | |
| 4 | 98.5 | 438.6 | 18.3 | 8496 | 41.4 | 24.7 | 6.3 | 1.26 |
| 5 | 93.9 | 382.8 | 28.8 | | 34.13 | 22.97 | 8.8 | 2.6 |
| | 96.3 | 383.5 | 27.0 | 4303 | | | | |
| 6 | 100 | 416.5 | 21.7 | | 39.47 | 27.99 | 8.8 | 1.98 |
| | 100 | 419.2 | 19.7 | 6343 | | | | |
| 7 | 100 | 654 | 15 | 5478 | 40.6 | 28.07 | 8.32 | 1.61 |
| 8 | 98.9 | 451.2 | 12.1 | | 37.7 | 32.15 | 11.1 | 1.9 |
| 9 | 100 | 530 | 19.8 | | 35.50 | 32.58 | 12.35 | 3.7 |
| | 100 | 521.5 | 15.5 | | 36.82 | 25.37 | 11.01 | 3.05 |
| 10 | 97.8 | 342.5 | 15.1 | 5653 | 44.7 | 24.9 | 10.1 | 1.65 |
| 11 | 98.9 | 451.2 | 12.1 | | 37.7 | 32.15 | 11.1 | 1.9 |
| 12 | 98.9 | 451.2 | 12.1 | | 37.7 | 32.15 | 11.1 | 1.9 |
| 13 | 100 | 452.6 | 13.3 | 7411 | 34.66 | 29.33 | 10.5 | 2.7 |
| 14 | 100 | 445.7 | 20.4 | 9970 | 37.65 | 28.72 | 8.66 | 1.77 |

TABLE 2
Effect of number of carbon atoms in olefin molecule on structure and characteristics of mixtures of non-fractionated
and non-hydrogenated oligo-olefins with boiling temperature above 170° C. when P ≅ 2 mm Hg

| | | | | | | Kinematic viscosity, cSt | | | Solidification | Flash |
|---|---|---|---|---|---|---|---|---|---|---|
| Examples | | CH₃ | C═C, | Mn, | d₄²⁰, | at | at | Viscosity | temperature, | point, |
| N^{o} | Olefins | 1̅0̅0̅0̅ C̅H̅₂̅ | mmole/g | g/mole | g/cm³ | 40° C. | 100° C. | index | ° C. | ° C. |
| 1 | Propylene | 2517 | 1.93 | 436 | 0.835 | 35.9 | 5.6 | 89 | −47 | 130 |
| 2 | | 2673 | 1.32 | 665 | 0.853 | 142.9 | 15.7 | 74 | −30 | 160 |
| 3 | | 2970 | 2.83 | 448 | 0.833 | 36.7 | 5.1 | 77 | −56 | 120 |
| 4* | | | 0.045 | 430 | 0.842 | 26.6 | 4.6 | 88 | −53 | — |
| 5 | Butene-1 | 1159 | 2.10 | 457 | 0.840 | 179.3 | 14.5 | 73 | −32 | — |
| 6 | | 1230 | 1.39 | 711 | 0.856 | 311.2 | 40.1 | 69 | −24 | 216 |
| 7 | | 1540 | 1.76 | 592 | 0.849 | 122.1 | 11.3 | 73 | −41 | — |
| 8 | Butene fraction | 1468 | 2.73 | 380 | — | 11.0 | 2.75 | 87 | −68 | — |
| 9 | | 1045 | 1.68 | 610 | — | 340.9 | 23.7 | 89 | −26 | — |
| 10 | | — | 2.00 | 500 | 0.842 | 187.4 | 17.2 | 98 | −45 | 120 |
| 11* | | 1250 | 0.046 | 500 | — | 204.4 | 17.4 | 91 | −43 | 124 |
| 12 | | — | 2.89 | 364 | 0.858 | 92.5 | 9.8 | 81 | −40 | 120 |
| 13** | | — | 0.02 | 450 | — | 78.8 | 10.0 | 48 | −25 | 198 |
| 14 | | 1293 | 0.80 | 1130 | 0.857 | 167.4 | 19.67 | 122 | −33.5 | 248 |
| 15 | Hexene-1 | 710 | — | — | — | 290 | 11 | 107 | −50 | 180 |
| 16 | | | — | — | — | 296 | 24 | 114 | −36 | 228 |
| 17 | Octene-1 | 470 | 1.65 | 600 | 0.835 | 35.4 | 6.4 | 130 | −50 | 192 |
| 18 | Decene-1 | 327 | 1.27 | 790 | 0.841 | 58.0 | 9.5 | 147 | −50 | 196 |
| 19 | Decene-5, trimer | 365 | 2.30 | 435 | 0.836 | 18.76 | 3.99 | 113 | −76 | 207 |
| 20 | LAO C₁₂-C₁₄ | 215 | 0.72 | 1180 | 0.849 | 120.0 | 17.0 | 132 | −38.5 | 253 |
| 21 | LAO C₁₂-C₁₄ | 212 | 0.73 | 1170 | 0.850 | 95.6 | 14.8 | 136 | −38.0 | 252 |

*Oligomers 4 and 11, respectively, upon hydrogenation;
**Oligomer 13 obtained in the atmosphere of toluene

TABLE 3
Effect of diverse factors on composition and structure of 1-Cl-dodecane
dechlorination products and decene-1 oligomers with aluminum

| | | 1-chloro- | | | | | | | |
|---|---|---|---|---|---|---|---|---|---|
| Example | Decene-1 | dodecane, | Aluminum | | RCl | Al | | τ, | CHROMATOGRAPHY % w |
| N^{o} | mole | mole | brand | mole | Al | R̅C̅l̅ | T, ° C. | min. | decene-1 |
| 1 | 0.089 | 0.0127 | PA-4 | 0.0255 | 0.5 | 2.0 | 20 | 60 | 71.5 |
| | | | | | | | 75 | 60 | 70.9 |
| | | | | | | | 95 | 60 | 71.35 |
| | | | | | | | 120 | 60 | 1.89 |
| 2 | 0.089 | 0.0127 | PA-1 | 0.00847 | 1.5 | 0.66 | 95 | 60 | 72.17 |
| | | | | | | | 120 | 60 | 2.39 |
| 3 | 0.089 | 0.0127 | PA-4 | 0.017 | 0.75 | 1.33 | 95 | 60 | 77.07 |
| | | | | | | | 120 | 60 | 76.2 |
| 4 | 0.089 | 0.0127 | PA-4 | 0.017 | 0.75 | 1.33 | 95 | 60 | 79.86 |
| | | | | | | | 120 | 60 | 0.88 |
| 5 | 0.089 | 0.0127 | PA-4 | 0.017 | 0.75 | 1.33 | 95 | 60 | 84.99 |
| | | | | | | | 115 | 60 | 2.79 |
| | | | | | | | 130 | 60 | 2.01 |
| 6 | PAO-NK | 0 | PA-4 | 0.0133 | 0 | 0 | 95 | 60 | 44.28 |
| | 20 ml | | | | | | 130 | 60 | 42.5 |
| | | | | | | | 130 | 120 | 43.99 |
| 7 | PAO-NK | 0 | PA-4 | 0.0133 | 0 | 0 | 95 | 60 | 44.7 |
| | 20 ml | | | | | | 130 | 60 | 44.7 |
| | | | | | | | 130 | 120 | 46.38 |
| 8 | 0.1055 | 0.0127 | PA-4 | 0.0254 | 0.54 | 1.8 | 95 | 60 | 23.25 |
| | | | | | | | 95 | 61 | 20.18 |
| | | | | | | | 95 | 60 | 3.06 |
| | | | | | | | 130 | 60 | 1.28 |
| 9 | 0.1055 | 0.0127 | PA-4 | 0.0127 | 1 | 1 | 95 | 60 | 8.31 |
| | | | | | | | 95 | 61 | 7.68 |
| | | | | | | | 95 | 60 | 6.30 |
| | | | | | | | 130 | 60 | 0 |

| | CHROMATOGRAPHY % w | | | | | | IKS, R/1000CH₂ | | |
|---|---|---|---|---|---|---|---|---|---|
| Example | | | | | | 1-chloro- | | trans- | |
| N^{o} | dimer | trimer | tetramer | pentamer | hexamer | dodecane | CH₃— | CH═CH | CH₂═CH |
| 1 | 4.59 | 3.4 | 0 | 0 | 0 | 20.11 | 221.2 | 6.9 | 76.8 |
| | 1.77 | 1.08 | 0 | 0 | 0 | 20.84 | 240.5 | 4.9 | 36.3 |
| | 2.34 | 2.66 | 0 | 0 | 0 | 20.90 | 229.3 | 6.5 | 70.3 |
| | 32.47 | 34.02 | 17.65 | 0 | 0 | 0 | 511.5 | 4.5 | 1.4 |
| 2 | 0.39 | 0 | 0 | 0 | 0 | 0 | 243.5 | 6.3 | 74.6 |
| | 40.48 | 31.29 | 9.61 | 0 | 0 | 0 | 569.9 | 4.9 | 2.1 |
| 3 | 0.86 | 0.43 | 0 | 0 | 0 | 21.44 | 229.6 | 5.6 | 73.9 |
| | 0 | 0 | 0 | 0 | 0 | 19.95 | 239.2 | 6.9 | 73.1 |
| 4 | 0.37 | 0.36 | 0.06 | 0 | 0 | 19.09 | 221.7 | 6.1 | 73.3 |
| | 25.35 | 52.1 | 18.6 | 0 | 0 | 2.27 | 480.5 | 11.2 | 0 |
| 5 | 0.19 | 0.16 | 0.05 | 0 | 0 | 14.4 | 202.0 | 7.08 | 29.0 |
| | 33.43 | 32.33 | 12.87 | 2.95 | 0 | 0 | 599.9 | 7.2 | 3.1 |
| | 34.23 | 31.81 | 13.22 | 3.25 | 0 | 0 | 572.8 | 5.2 | 0 |
| 6 | 11.1 | 18.4 | 12.72 | 6.63 | 1.07 | 0 | 356.6 | 7.2 | 41.6 |
| | 11.08 | 18.78 | 12.97 | 6.63 | 1.05 | 0 | 376.2 | 6.5 | 37.8 |
| | 11.18 | 19.17 | 12.86 | 6.61 | 1.1 | 0 | 361.6 | 6.5 | 39.5 |
| 7 | 10.52 | 17.5 | 12.24 | 6.39 | 0 | 0 | 362.7 | 7.4 | 43.0 |
| | 10.21 | 16.9 | 11.75 | 5.92 | 1.08 | 0 | 391.8 | 7.9 | 42.7 |
| | 10.78 | 19.56 | 13.47 | 6.85 | 11.16 | 0 | 363.6 | 6.9 | 37.2 |
| 8 | 20.33 | 28.07 | 18.55 | 9.46 | 1.80 | 0 | 376.1 | 10.7 | 0 |
| | 11.89 | 18.08 | 12.91 | 6.53 | 0 | 16.65 | 318.9 | 9.2 | 1.1 |
| | 20.74 | 19.15 | 12.98 | 12.77 | 0 | 2.38 | 456.8 | 2.0 | 0 |
| | 20.36 | 17.82 | 12.27 | 11.15 | 0 | 0.21 | 455.6 | 1.8 | 0 |
| 9 | 6.04 | 56.94 | 22.29 | 13.50 | 0 | 0 | 283.6 | 8.0 | 0 |
| | 11.67 | 18.17 | 13.37 | 7.28 | 2.2 | 28.2 | 241.3 | 6.3 | 0 |
| | 10.77 | 17.99 | 14.35 | 8.27 | 2.9 | 28.5 | 232.9 | 5.6 | 0 |
| | 9.66 | 15.50 | 10.00 | 7.40 | 0 | 0 | 356.7 | 12.4 | 0 |

In experiments 8 and 9 pre-polymerization of 20 ml decene-1 was carried out under action of system EASCh (00008 mole) − TBCh (0.0012 mole) at 95° C.

TABLE 4
Effect of diverse factors on composition and structure of 1-Cl-dodecane dechlorination
products and decene-1 oligomers with triethylaluminum (TEA)

| | | 1-chloro- | | | | | | | |
|---|---|---|---|---|---|---|---|---|---|
| Example | Decene-1, | dodecane, | AOC | | RCl | Al | | τ, | CHROMATOGRAPHY, % w |
| N^{o} | mole | mole | nature | mole | Al | R̅C̅l̅ | T, ° C. | min. | decene-1 |
| 1 | PAO-NK | 0.0127 | AlEt₃ | 0.0254 | 0.5 | 2.0 | 20 | 60 | 46.02 |
| | 20 ml | | | | | | 75 | 60 | 45.83 |
| | | | | | | | 95 | 60 | 47.2 |
| | | | | | | | 130 | 60 | 41.7 |
| 2 | 0.1055 | TBCh* | EASX | 0.0008 | 1.5 | 0.67 | 95 | 60 | 17.0 |
| | | 0.0012 | | | | | | | |
| | | 0.0127 | AlEt₃ | 0.0254 | 0.5 | 2.0 | 95 | 60 | 3.7 |
| 3 | 0.1055 | TBCh* | EASX | 0.0008 | 1.5 | 0.67 | 95 | 60 | 13.01 |
| | | 0.0012 | | | | | | | |
| | | 0.0127 | AlEt₃ | 0.0254 | 0.5 | 2.0 | 95 | 60 | 2.84 |
| | | | | | | | 130 | 120 | 2.08 |
| 4 | 0.1055 | TBCh* | EASX | 0.0008 | 1.5 | 0.67 | 95 | 60 | 14.7 |
| | | 0.0012 | | | | | | | |
| | | 0.0127 | AlEt₃ | 0.0254 | 0.5 | 2.0 | 95 | 61 | 7.78 |
| | | | | | | | 130 | 60 | 2.91 |
| 5 | 0.1055 | TBCh* | EASX | 0.0008 | 1.5 | 0.67 | 95 | 60 | 12.26 |
| | | 0.0012 | | | | | | | |
| | | 0.0127 | AlEt₃ | 0.0125 | 1.0 | 1.0 | 95 | 61 | 10.26 |
| | | | | | | | 130 | 60 | 2.45 |
| 6 | PAO-NK | 0.064 | AlEt₃ | | 1.0 | 1.0 | 20 | 1 | 46.48 |
| | 85 ml | | | | | | 164 | 60 | 0 |
| 7 | 0.446 | 0.064 | AlEt₃ | 0.064 | 1.0 | 1.0 | 150 | 1 | 80.0 |
| | | | | | | | 150 | 60 | 0 |

| | CHROMATOGRAPHY, % w | | | | | | IKS, R/1000CH₂ | | |
|---|---|---|---|---|---|---|---|---|---|
| Example | | | | | | 1-chloro- | | trans- | |
| N^{o} | dimer | trimer | tetramer | pentamer | hexamer | dodecane | CH₃— | CH═CH | CH₂═CH |
| 1 | 5.67 | 8.4 | 5.79 | 2.76 | 1.18 | 18.8 | 305.7 | 4.1 | 38.8 |
| | 6.17 | 9.6 | 6.9 | 3.52 | 0.99 | 19.6 | 285.4 | 3.3 | 30.5 |
| | 5.81 | 9.54 | 6.5 | 3.35 | 1.15 | 18.3 | 279.1 | 4.0 | 39.2 |
| | 7.07 | 11.4 | 7.89 | 4.02 | 1.17 | 0.3 | 288.7 | 4.7 | 31.5 |
| 2 | 11.19 | 21.43 | 16.19 | 11.56 | 3.8 | 0.15 | 333.6 | 6.2 | 6.4 |
| | 18.83 | 22.10 | 15.79 | 11.4 | 0 | 0 | 381.4 | 4.3 | 0 |
| 3 | 15.95 | 24.67 | 20.8 | 11.63 | 0 | 0 | 369.5 | 8.8 | 0 |
| | 29.39 | 21.27 | 12.74 | 6.33 | 0 | — | 426.7 | 2.9 | 4.8 |
| | 30.59 | 23.36 | 13.88 | 5.9 | 0 | — | 421.3 | 1.9 | 1.9 |
| 4 | 15.41 | 24.57 | 16.5 | 10.17 | 3.35 | 0 | 355.9 | 8.1 | 0 |
| | 8.93 | 17.5 | 12.1 | 7.15 | 1.78 | 32.5 | 287.3 | 5.8 | 0 |
| | 22.71 | 19.38 | 12.55 | 10.44 | 0 | 0.65 | 443.8 | 0.67 | 0 |
| 5 | 22.5 | 20.73 | 16.3 | 11.45 | 2.85 | 0 | 339.6 | 6.1 | 2.9 |
| | 8.3 | 17.77 | 14.06 | 9.91 | 2.01 | 29.06 | 282.0 | 4.4 | 1.7 |
| | 19.91 | 19.19 | 12.74 | 0 | 7.47 | 21.41 | 414.9 | 1.9 | 0 |
| 6 | 11.89 | 13.52 | 9.69 | 0 | 4.57 | 12.0 | 292.9 | 6.2 | 28.5 |
| | 39.3 | 27.26 | 14.26 | 5.59 | 0 | 0 | 379.3 | 1.5 | 0 |
| 7 | 1.08 | 0.87 | 0 | 0 | 0 | 15.1 | 250.2 | 71.0 | 78.6 |
| | 25.4 | 14.38 | 6.9 | 28.9 | 0 | 0 | 326.6 | 0 | 0 |

Note:
PAO-NK - mixture of oligomerizate from the Nizhnekamsk plant of synthetic oils with decene-1;
EASX - ethylaluminum chloride;
*in these experiments first carried out was decene-1 oligomerization under action of system EASX-TBCh, followed by addition of 1-chloro-dodecane and triethylaluminum; AOC - organoaluminum compound; RCl - chloralkanes-TBCh and 1-chloro-dodecane.

TABLE 5
Effect of diverse factors on composition and structure of 1-Cl-dodecane dechlorination products
and decene-1 oligomers with KOH/NaOH solutions in n-butyl, hexyl and other alcohols

| | | 1-chloro- | | | | | | | |
|---|---|---|---|---|---|---|---|---|---|
| Example | Decene-1, | dodecane, | ROH | | KOH | KOH/ | | τ, | CHROMATOGRAPHY |
| N^{o} | mole | mole | nature | mole | mole | RCl | T, ° C. | min. | decene-1 |
| 1 | 0.0527 | 0.0127 | n-C₄H₉OH | 0.1092 | 0.058 | 4.5 | 120 | 60 | 65.12 |
| | | | | | | | 120 | 24 h. | 59.0 |
| 2 | 0.0527 | 0.0127 | n-C₄H₉OH | 0.1093 | 0.058 | 4.5 | 120 | 5 min. | 54.12 |
| | | | | | | | 120 | 2 h. | 70.5 |
| | | | | | | | 120 | 4 h. | 69.6 |
| | | | | | | | 120 | 7 h. | 67.3 |
| | | | | | | | 120 | 27 h. | 63.7 |
| 3 | 0.0527 | 0.0127 | C₆H₁₃OH | 0.0802 | 0.058 | 4.5 | 20 | 10 min. | 79.64 |
| | | | | | | | 130 | 2 h. | 46.8 |
| | | | | | | | 130 | 4 h. | 46.11 |
| 4 | 0.0527 | 0.0127 | C₆H₁₃OH | 0.0802 | NaOH | | 20 | 10 | 45.68 |
| | | | | | 0.08 | 6.3 | 130 | 120 | 64.41 |
| | | | | | | | 130 | 120 | 66.26 |
| | | | AFTER 24 HOURS | | | | 130 | 180 | 32.2 |
| 5 | 0.527 | 0.127 | n-C₄H₉OH | 0.802 | 0.577 | 4.5 | 20 | 60 | 62.59 |
| | | | | | | | 150 | 60 | 52.76 |

| | |
|---|---|
| CHROMATOGRAPHY | IKS, R/1000CH₂ |

| Example | | | | | | 1-chloro- | | trans | |
|---|---|---|---|---|---|---|---|---|---|
| N^{o} | dimer | trimer | tetramer | pentamer | hexamer | dodecane | CH₃— | CH═CH | CH₂═CH |
| 1 | 15.31 | 0.16 | 0 | 0 | 0 | 16.8 | 284.9 | 0 | 75.5 |
| | 11.12 | 0.02 | 0 | 0 | 0 | 11.8 | 360.3 | 14.6 | 31.8 |
| 2 | 1.99 | 0 | 0 | 0 | 0 | 14.0 | 452.6 | 16.2 | 56.0 |
| | 18.7 | 0.27 | 0 | 0 | 0 | 7.9 | 181.7 | 1.9 | 34.8 |
| | 21.8 | 0.33 | 0 | 0 | 0 | 4.3 | 270.6 | 6.8 | 107.2 |
| | 26.7 | 0.68 | 0 | 0 | 0 | 4.2 | 214.3 | 5.6 | 33.7 |
| | 28.6 | 1.60 | 0 | 0 | 0 | 4.8 | 253.8 | 0 | 121.5 |
| 3 | 2.39 | 0 | 0 | 0 | 0 | 11.91 | 351.5 | 7.4 | 80.6 |
| | 20.6 | 0.36 | 0 | 0 | 0 | 0.986 | 293.0 | 0 | 69.3 |
| | 19.63 | 0 | 0 | 0 | 0 | 0 | | | |
| 4 | 8.45 | 0.30 | 0 | 0 | 0 | 12.0 | 202.5 | 4.3 | 41.9 |
| | 14.42 | 1.26 | 0 | 0 | 0 | 11.9 | 179.6 | 0.84 | 39.99 |
| | 17.6 | 1.16 | 0 | 0 | 0 | 10.92 | 250.6 | 6.3 | 55.2 |
| | 28.57 | 1.59 | 0 | 0 | 0 | 2.52 | 306.7 | 7.99 | 56.8 |
| 5 | 1.37 | 0.55 | 0 | 0 | 0 | 19.94 | 348.4 | 12.9 | 35.9 |
| | 28.37 | 0.71 | 0 | 0 | 0 | 0.47 | 398.2 | 18.4 | 42.3 |

TABLE 6
Results of thermal decene oligomerizate dehydrochlorination comprising
735 ppm monoorganochlorine compounds (0.0735% w chlorine)

| | | | | | | | | | | | Residue |
|---|---|---|---|---|---|---|---|---|---|---|---|
| | | | | Retort, | Chlorine content | | | Chlorine content | | Chlorine, | solidification |
| Example | Oligomer | Cl starting | Reaction | residue, | in residue | | Blowing, | in blowing | | found, | temperature, |
| N^{o} | load, g | content, g | time, hours | g | ppm | g | g | ppm | g | % w | T ° C. |
| 1 | 255 | 0.1874 | 2 | 189 | 23 | 0.0043 | 66 | | | | <−60 |
| 2 | 255 | 0.1874 | 3 | 192 | 20 | 0.0038 | 63 | 1333 | 0.0840 | 96.10 | <−60 |
| 3 | 276 | 0.2032 | 8 | 211 | 24 | 0.0051 | 65 | | | | <−60 |
| 4 | 294 | 0.2161 | 7 | 222 | 42 | 0.0093 | 72 | 1303 | 0.0938 | 94.70 | <−60 |
| 5 | 276 | 0.2029 | 1 | 204 | 11 | 0.0022 | 72 | | 0.0621 | | −64 |
| 6 | 309 | 0.2271 | 2 | 217 | 4 | 0.0009 | 92 | 1625 | 0.1495 | 90.12 | −54 |
| 7 | 276 | 0.2029 | 2 | 202 | 0 | 0 | 74 | 1480 | 0.1095 | 92.13 | — |
| 8 | 247 | 0.1815 | 1 | 187 | 3 | 0.0006 | 60 | 1599 | 0.0959 | 97.94 | −60 |

Note:
Thermal chlorine-containing oligodecene dehydrochlorination in Examples 3 and 4 was carried out at 300° C.;
in Example 2 - at 330° C.;
in Example 1 at 340° C. under static conditions without HCl blowing with nitrogen;
thermal dehydrochlorination of chlorine-containing oligodecenes in Examples 5-8 was conducted at 330° C. under the conditions of blowing HCl and other chlorine-containing components with nitrogen (blowing rate 3 l/h).

TABLE 7
Thermal dehydrochlorination of chlorinecontaining oligodecenes in the presence of alkali (MOH).
The chlorine content in initial oigomerizate - 280 ppm. Reaction duration - 60 min.

| Oligomer | MOH | | | | | Chlorine content in product, ppm | | | | |
|---|---|---|---|---|---|---|---|---|---|---|
| weight, | | weight, | MOH dispersing agent | | T, | initial | washed | | solid | distilled |
| g | nature | g | nature | weight | ° C. | oligomer | oligomer | wash H₂O | residue | H₂O |
| 250 | KOH | 5 | H₂O | 20 | 300 | 180 | 145 | 0 | 3372 | 0 |
| 250 | KOH | 5 | H₂O | 20 | 310 | 180 | 79 | 11 | 3549 | 0 |
| 250 | KOH | 5 | H₂O | 20 | 320 | 180 | 35 | 17 | 3905 | 0 |
| 250 | KOH | 5 | H₂O | 20 | 330 | 180 | 8 | 14 | 4685 | 0 |
| 250 | KOH | 5 | H₂O | 20 | 330 | 4305 | 35 | 55 | 2.12% | 0 |
| 250 | NaOH | 5 | H₂O | 20 | 330 | 180 | 79 | 7 | 5199 | 0 |
| 250 | KOH | 5 | C₂H₅OH | 20 | 330 | 180 | 62 | 7 | 4188 | — |
| 125 | KOH | 5 | n-C₄H₉OH | 125 | 200 | 180 | 110 | 32 | — | — |

TABLE 8
Dehydrochlorination of chloroparaffins (ChP) containing 43 w. Chlorine with KOH solutions in n-butanol under
conditions of azeotropic distillation of water during reaction. Initial C₄H₉OH/C—Cl mole ratio = 2.7.

| | | Reaction | output | DHChP | Dehydrochloroparaffin characteristic (DHChP) | | | | | | |
|---|---|---|---|---|---|---|---|---|---|---|---|
| KOH/C—Cl, | Reaction | time, | H₂O, mole/ | output, | Cl content | | MM, | Br number, g | C═C | C₄H₉O in molecule | |
| mole/mole | T ° C. | hrs | mole CCl | % w | % w | mole/mole | g/mole | Br₂/100 g | in molecule | % w | mole/mole |
| 1.25 | 120 | 3 | — | 72.2 | 8.53 | 1.45 | 600 | 115.0 | 4.3 | 10.8 | 0.89 |
| 1.25 | 107 | 3 | — | 76.0 | 13.5 | 1.9 | 520 | 93.1 | 3.0 | 8.9 | 0.64 |
| 1.25 | 121 | 3 | 1.34 | 74.9 | 12.9 | 1.8 | 490 | 109.0 | 3.4 | 9.8 | 0.66 |
| 1.25 | 108 | 6 | — | 74.5 | 11.4 | 1.7 | 533 | 108.0 | 3.6 | 10.6 | 0.65 |
| 1.25 | 122 | 6 | 1.56 | 73.3 | 6.3 | 0.9 | 508 | 128.6 | 4.1 | 9.3 | 0.77 |
| 1.25 | 140 | 7 | 1.61 | 69.0 | 3.74 | 0.6 | 533 | 144.0 | 4.8 | 11.7 | 0.81 |
| 1.10 | 140 | 7 | 1.47 | 69.0 | 3.83 | 0.5 | 505 | 135.5 | 4.3 | — | — |
| 1.00 | 140 | 7 | 1.50 | 68.2 | 4.08 | 0.6 | 513 | 135.0 | 4.3 | 13.3 | 0.93 |
| 0.98 | 140 | 7 | 1.50 | 70.0 | 6.25 | 0.9 | 535 | 125.0 | 4.2 | 12.5 | 0.92 |

TABLE 9
Results of separation of decene oligomerizate into fractions under conditions of partial
depolymerization of high-molecular oligodecenes and characteristic of fractions

| | | Oligomerizate makeup | | | | | | | | | | |
|---|---|---|---|---|---|---|---|---|---|---|---|---|
| | Oligo- | | dimers (D) | | trimers (T) | | still residue (K) | | Fraction characteristics | | | |
| Example | merizate | decenes, | (PAO-2) | | (PAO-4) | | (PAO-10+) | | C═C, | Mₙ, | kinematic | |
| N^{o} | in still, g | g | g | % w | g | % w | g | % w | mmole/g | g/mole | viscosity, cSt | I.V. |
| 1 | 7100 | 130 | 415 | 5.8 | 2765 | 38.9 | 3550 | 50.0 | [symbol] : 3.79 | 264 | 2.08 | — |
| | | | | | | | | | T: 2.32 | 430 | 4.09 | 133 |
| | | | | | | | | | K: 1.25 | 800 | 12.93 | 130 |
| 2 | 8800 | 215 | 380 | 4.3 | 4755 | 54.0 | 2610 | 29.7 | T: 2.41 | 415 | 3.96 | 129 |
| 3 | 8565 | 170 | 585 | 6.8 | 4320 | 50.4 | 3350 | 39.1 | T: 2.16 | 462 | 4.21 | 136 |
| 4 | 8565 | 120 | 1575 | 18.4 | 4285 | 50.0 | 2430 | 28.4 | — | — | — | — |
| 5 | 8600 | 160 | 160 | 16.9 | — | — | 6970 | 81.0 | K: 1.50 | 660 | 9.82 | 124 |
| 6 | 8635 | 90 | 80 | 18.2 | — | — | 6900 | 79.9 | K: 1.39 | 716 | 10.54 | 127 |

Note:
Dimers are released in heating a still to 300° C. and column's head - to 180° C.; trimers are released in heating a still to 350° C. and column's head - 230° C. at a residual pressure of 1 mm Hg.

TABLE 10
Output, composition, structure and properties of products obtained
by thermal directed depolymerization of oligodecene (with a kinematic
viscosity of 100° C. = 10 cSt) at 360° C.

| | | | Fraction composition | | | | | |
|---|---|---|---|---|---|---|---|---|
| | Fraction, | | | | Tetra- | | | |
| ΔT, | yield | | Dimers, | Trimers, | mers, | Still, | IKS, R/1000 CH₂ | |
| ° C. | g | % w | % w | % w | % w | % w | CH₃ | CH₂═CH— |
| 20-150 | 23 | 8.0 | — | — | — | — | — | 53.6% |
| 150-240 | 34 | 12.0 | 99.0 | — | — | — | | |
| 240-300 | 42 | 15.0 | 3.0 | 96.0 | 1.0 | — | Before hydrogenation | |
| 240-300 | 42 | 15.0 | 3.0 | 96.0 | 1.0 | — | After hydrogenation | |
| 300-310 | 76 | 1.85 | 0.7 | 89.0 | 10.3 | — | 350.4 | — |
| 310-320 | 92 | 2.24 | 0.6 | 58.8 | 40.8 | — | 352.3 | — |
| 320-330 | 182 | 4.44 | 0.5 | 39.1 | 60.4 | — | 315.6 | — |
| 330-340 | 90 | 2.20 | 0.5 | 6.5 | 93.0 | — | 344.5 | 4.2 |
| 340-350 | 286 | 6.98 | — | — | — | — | 317.6 | — |

| | | | | | | | |
|---|---|---|---|---|---|---|---|
| >360 | 286 | 32.0 | — | — | — | 32.0 | Before hydrogenation |
| >360 | 286 | 32.0 | — | — | — | 32.0 | After hydrogenation |

| | IKS, R/1000 CH₂ | | Properties of products | | | | |
|---|---|---|---|---|---|---|---|
| ΔT, | trans- | | Tsolid, | Tflash | η₄₀, | η₁₀₀, | |
| ° C. | —CH═CH— | CH₂═C< | ° C. | point° C. | cSt | cSt | I.V. |
| 20-150 | 18.1% | 28.3% | — | — | — | — | — |
| 150-240 | | | −17 | 148 | 4.83 | 1.74 | — |
| 240-300 | Before hydrogenation | | −38 | 206 | 14.54 | 3.53 | 130 |
| 240-300 | After hydrogenation | | −37 | 106 | 16.39 | 3.83 | 135 |
| 300-310 | 7.9 | 2.2 | −72 | | 15.67 | 3.69 | 129 |
| 310-320 | 7.8 | 2.5 | — | 220 | 16.40 | 3.84 | 135 |
| 320-330 | 6.3 | 2.9 | −52 | 180 | 20.20 | 4.46 | 141 |
| 330-340 | 6.6 | 5.1 | −50 | | 18.10 | 4.19 | 147 |
| 340-350 | 4.0 | 2.7 | — | — | — | — | — |

| | | | | | | |
|---|---|---|---|---|---|---|
| >360 | Before hydrogenation | −62 | 256 | 44.85 | 7.72 | 131 |
| >360 | After hydrogenation | −60 | 248 | 62.53 | 9.70 | 127 |

Note:
ΔT - temperature range in which a fraction is released from products of depolymerization

1. A method for the preparation of polyolefinic bases of synthetic oils by the oligomerization of higher olefins, comprising the steps of conditioning olefinic feedstock and the solutions of components of a cationic catalytic system, isomerizing higher linear alpha-olefins, oligomerizing said olefinic feedstock under the action of a cationic aluminum-containing catalytic system, separating spent catalyst out of an oligomerizate, separating the oligomerizate into fractions and hydrogenating the fractions separated, in which a step of dechlorination of monochlorine-containing oligomers present in the oligomerizate is carried out after the step of oligomerization and/or after the step of separation of spent catalyst from the oligomerizate and after the step of separation of the oligomerizate into fractions is carried out a step of depolymerization of high-molecular products separated as still bottoms in the step of separation of the oligomerizate into fractions.

2. The method according to claim 1, wherein the oligomerization of higher olefins is carried out in the mixtures of oligomerizable higher olefins with the products of oligomerization thereof or in the mixtures of oligomerizable higher olefins with the products of oligomerization thereof and with aromatic hydrocarbons under the action of a catalytic system Al(O)—HCl—(CH₃)₃CCl at temperatures of from 110 tp′180° C., in Al(O) concentrations of from 0.02 to 0.08 g-atom/l, at HCl/Al(O) mole ratios variable in the range of from 0.002 to 0.06 and RCl/Al(O) mole ratios being variable in the range of 1.0 to 5.0, wherein the Al(O) is highly dispersed powdery arαaminum having a particle size varying in the range of from 1 to 100 mem, for example Al(O), PA-I, PA-4, ACD-4, ACD-40, ACD-T, PAP-I brands.

3. The method according to claim 1, wherein the higher olefins taken are represented by the mixtures of linear or branched alpha-olefins with iso-olefins and olefins with the intramolecular arrangement of a double bond (with “inner” olefins) containing from 4 to 14 (predominantly 10) carbon atoms, with the following ratio of ingredients, % w:alpha-olefins 0.5-99.0; iso-olefins 0.5-5.0; “inner” olefins—the balance up to 100% w.

4. The method according to claim 1, wherein the dechlorination of oligomerizate-present monochlorine-containing oligomers (RCl) is carried out after the step of oligomerization with highly dispersed powdery metallic aluminum —Al(O) having a particle size varying in the range of from 1 to 100 mem (for example, PA-I, PA-4, ACD-4, ACD-40, ACD-T, PAP-I brands) with Al(O)/RCl mole ratios varying in the range of from 0.5 to 2.0 in the temperature range of from 110 to 180° C. for 30 to 180 minutes.

5. The method according to claim 1, wherein the dechlorination of oligomerizate-present mono chlorine-containing oligomers (RCl) is carried out after the step of oligomerization with triethyl aluminum (TEA) with TEA/RCl mole ratios varying in the range of from 0.5 to 2.0, in the temperature range of from 95 to 150° C. for 30 to 180 minutes.

6. The method according to claim 1, wherein the dechlorination of oligomerizate-present monochlorine-containing oligomers (RCl) is carried out after the step of separation of spent catalyst with the alcohol solution of a potassium/sodium hydroxide (MOH) with MOH/RCl mole ratios varying in the range of 1.1 to 2.0, in the temperature range of from 120 to 160° C. for 30 to 240 minutes.

7. The method according to claim 1, wherein the dechlorination of oligomerizate-present monochlorine-containing oligomers (RCl) is carried out after the step of separation of spent catalyst by thermal dehydrochlorination thereof in the temperature range of from 280 to 350° C. and at pressures of 1-2 bar for 30 to 180 minutes in blowing an escaping hydrogen chloride with nitrogen, carbon dioxide, methane or overheated water vapor.

8. The method according to claim 1, wherein the dechlorination of oligomerizate-present monochlorine-containing oligomers (RCl) is carried out in the presence of dry alkali metal hydroxides (MOH) with MOH/RCl mole ratios varying in the range of 1.1 to 2.0.

9. The method according to claim 8, wherein the dry alkali metal hydroxides are obtained directly in the oligomerizate by the distillation of water on heating a mixture of the spent catalyst-free oligomerizate and 5-40% aqueous solution of an alkali metal hydroxide at temperature varying in the temperature range of from 100 to 200° C.

10. The method according to claim 1, wherein subjected to dechlorination are mono-, di- and polychlorine-containing aliphatic and aromatic hydrocarbons, oligomers, polymers, oil fractions and liquid or solid waste materials.

11. The method according to claim 1, wherein the depolymerization of high-molecular products separated out in the form of still bottoms in the step of separation of the oligomerizate into fractions is carried out by heating same at temperatures varying in the range of from 330 to 360° C. and at pressures of from 1.0 to 10.0 mm Hg for 30 to 120 minutes while continuously removing the products from a depolymerization reactor.

12. The method according to claim 1, wherein the hydrogenation of oligo-olefin narrow fractions separated out of the oligomerizate is carried out under the action of a palladium alumina-supported catalyst (predominantly —Pd(0.2% w)/Al₂θ?3) modified with anhydrous potassium hydroxide which is taken in an amount of from 30 to 100% w, as calculated for hydrogenation catalyst at temperatures varying in the range of 150 to 200° C. and a hydrogen pressure of 20 at.
